(19) United States
(12) Patent Application Publication
(10) Pub. No.: US 2007/0253997 A1
(43) Pub. Date: Nov. 1, 2007

(54) Drug delivery to alleviate chronic pelvic pain

(75) Inventors: Jonathon E. Giftakis, Maple Grove, MN (US); Martin T. Gerber, Maple Grove, MN (US)

Correspondence Address: SHUMAKER & SIEFFERT, P. A., 1625 RADIO DRIVE, SUITE 300, WOODBURY, MN, 55125, US

(73) Assignee: Medtronic, Inc.

(21) Appl. No.: 11/413,617

(22) Filed: Apr. 28, 2006

(51) Int. Cl. A61K 38/08; A61K 31/485; A61K 31/195; A61K 31/192; A61N 1/00

(52) U.S. Cl. 424/422; 607/40; 514/282; 514/317; 514/561; 514/16; 514/524

(57) ABSTRACT

The disclosure describes a method and system for delivering a drug to a genitofemoral nerve or a genital branch of a genitofemoral nerve of a patient. The system includes drug delivery devices that deliver one or more drugs for alleviation of pelvic pain. The system may deliver drug therapy for pelvic pain in men or women. Drug therapy may be delivered at various locations along a single or both genitofemoral nerves and the genital branch of a single or both genitofemoral nerves of a patient via a fluid transfer device. When implanted proximate to a genital nerve branch, the fluid transfer device may be implanted proximate to the genital nerve branch. In a male patient stimulation may be delivered proximate to the spermatic cord, which contains a portion of the genital nerve branch. In some embodiments, electrical stimulation may be applied in combination with drug therapy to a genitofemoral nerve or a genital nerve branch of the genitofemoral nerve of a patient.

### TECHNICAL FIELD

The invention relates to medical devices and, more particularly, to devices for delivering neuromodulation therapy.

### BACKGROUND

Pain in the pelvic region, including urogenital pain, may be caused by a variety of injuries or disorders in men and women. For example, chronic testicular pain (CTP), post vasectomy pain, genitofemoral neuralgia and other pain originating from the testicles, groin, or abdomen are common reasons for referral to a urological specialist. The incidence of patients with CTP, also referred to as orchialgia, orchidynia, or chronic scrotal pain, is large and may be caused by on-going inflammation of the testicle (orchitis) or epididymis (epdidymitis), trauma, tumors, hernia, torsion (twisting of the testicle), varicocele, hydrocele, spermatocele polyarteritis nodosa, and previous surgical interventions such as vasectomy and hernia surgery.

As an example, CTP or genitofemoral neuralgia may be attributed to nerve injury, such as stretching of a nerve, electrocoagulation, stricture caused by ligation, entrapment of the nerve in scar tissue, or irritation because of proximity to a zone of inflammation, during inguinal herniorrhaphy. The pain experienced by the patient may be unilateral or bilateral, constant or intermittent, spontaneous or exacerbated by physical activities and pressure, and may remain localized in the scrotum or radiate to the groin, perineum, back, or legs.

Typically, testicle removal and spermatic cord denervation procedures are used to treat CTP. In spermatic cord denervation procedures, nerves in or adjacent to the spermatic cord, i.e., the genitofemoral nerve or sympathetic nerves, are severed or permanently removed. Such procedures may result in permanent and substantial pain relief regardless of the origin of pain. However, severing or removing these nerves may result in loss of sensation in the testicle and/or scrotum, loss of the cremasteric reflex which may cause fertility issues, and even loss of blood flow causing the testicle to die. Therapeutic nerve blocks may also be used to treat CTP, but generally only relieve pain temporarily.

In addition, women may experience various types of sources of pelvic pain. Sources of pain may include injury to nerves resulting from surgical procedures, non-surgical conditions, vulvodynia which can be very debilitating but has no obvious source, and interstitial cystitis (painful bladder syndrome). Interstitial cystitis may be a source of pelvic pain in both women and men. Surgical procedures that may injure nerves in the pelvic region may include urological operations in the pelvic area, gynecological surgery, and hysterectomy. Non-surgical conditions which cause pain in women include adhesions, endometriosis, and pelvic congestion.

### SUMMARY

In general, the invention is directed to techniques for delivering a drug to a genitofemoral nerve or a genital nerve branch of the genitofemoral nerve of a patient via an implantable drug delivery device to alleviate symptoms of chronic pelvic pain in men or women. Pelvic pain may include urogenital pain or other forms of pelvic pain. The drug may be delivered to one or both genitofemoral nerves. In addition, the drug may be applied directly to the genital branch of one or both genitofemoral nerves or, in the case of male patients, indirectly via the spermatic cord which contains a portion of the genital nerve branch. In some embodiments, electrical stimulation may be applied in combination with drug delivery to a genitofemoral nerve or a genital nerve branch of the genitofemoral nerve of a patient.

A system according to the invention may include a drug delivery device, e.g., an implantable drug pump, that delivers a drug or, in some embodiments, more than one drug, to the genitofemoral nerve or the genital branch of the genitofemoral nerve, e.g. via the spermatic cord in a male patient, to alleviate chronic testicular pain (CTP) or other afflictions associated with pelvic pain, including pain originating from the testicles, groin, or abdomen, such as post vasectomy pain and genitofemoral neuralgia. In female patients, the drug delivery device delivers the drug to the genitofemoral nerve or genital nerve branch to alleviate other types of pelvic pain such as vulvodynia, interstitial cystitis, post-operative pain, adhesions, endometriosis or pelvic congestion.

The drug delivery device may comprise a reservoir for storing a drug, one or more fluid transfer devices, such as a catheter, a conduit, or the like, to transfer the drug from the reservoir to the delivery site, and a pump coupling the reservoir to the fluid transfer devices that pumps the drug from the reservoir to the delivery site via the fluid transfer devices. In some embodiments, the drug delivery device may be capable of delivering one or more drugs and, accordingly, may include more than one reservoir. Each reservoir may contain a drug or a mixture of drugs. The drug delivery device may also include a processor that controls the function of the drug delivery device to, for example, control which of the plurality of drugs contained in the drug delivery device are delivered and the dosage of the drugs delivered.

The fluid transfer devices may be implanted at various locations proximate to one or both of the genitofemoral nerves of a patient or the genital branch of one or both genitofemoral nerves. Additionally or alternatively, a fluid transfer device may be implanted proximate to the spermatic cord to deliver the drug indirectly to the genital nerve branch. In this manner, the drug may be delivered unilaterally (to one cord or branch) or bi-laterally (to both cords or branches).

For male patients, fluid transfer devices may be implanted using well known surgical procedures for exposing the spermatic cord, e.g., inguinal incision as used for spermatic cord denervation or hernia repair. Systems including such fluid transfer mechanisms and employing the techniques described in this disclosure may substantially reduce or eliminate chronic pelvic pain, including urogenital pain such as CTP, without loss of sensation in the testicles and/or scrotum or loss of the cremasteric reflex as is common with testicle removal and spermatic cord denervation procedures.

In some embodiments, electrical stimulation may be applied in combination with drug delivery. Accordingly, a system according to the invention may include, in addition to a drug delivery device, one or more electrical stimulators that apply electrical stimulation to the genitofemoral nerve or the genital branch of the genitofemoral nerve, e.g., via the spermatic cord in a patient, to alleviate CTP or other afflictions associated with pelvic pain in men and women. The electrical stimulators may comprise various types of electrodes such as cuff electrodes, ring electrode leads, paddle leads, and/or microstimulators implanted at various locations proximate to one or both of the genitofemoral nerves of a patient or the genital branch of one or both genitofemoral nerves to apply stimulation uni-laterally or bi-laterally.

The electrical stimulators may be coupled to an implantable stimulation device implanted within a subcutaneous pocket in the abdomen of the patient or, alternatively, the scrotum or buttock of the patient. The implantable stimulation device may be incorporated with the drug delivery device in a single device, e.g., an implantable medical device, or may be independent of the drug delivery device. In any case, the electrical stimulators may be coupled to the stimulation device via standard electrode leads. The electrical stimulators may be capable of wireless communication with other implantable medical devices, an external programmer, or both.

Systems according to the invention may include an external programmer that programs the drug delivery device to deliver one or more drugs to a genitofemoral nerve or genital nerve branch, e.g., directly or via a respective spermatic cord. During drug delivery, a clinician or patient may operate the external programmer to adjust delivery parameters, such as which of the plurality of drugs contained in the device are delivered and the dosage of the drugs delivered. In some cases, a patient may use the programmer to deliver a drug on demand, e.g., when the patient experiences discomfort. Additionally, or alternatively, the drug delivery device may store drug delivery programs and schedules. In this manner, the drug can be delivered according to preprogrammed parameters and schedules, if desired.

In embodiments in which the system delivers electrical stimulation in combination with a drug, a clinician or patient may similarly operate the external programmer to adjust stimulation parameters and/or deliver stimulation on demand. In such embodiments, the implantable stimulation device may store stimulation programs and schedules and deliver stimulation according to preprogrammed stimulation parameters and schedules.

In one embodiment, the invention provides a method comprising delivering a drug to a genital nerve branch of a genitofemoral nerve of a patient via an implanted drug delivery device.

In another embodiment, the invention provides a system comprising an implantable drug delivery device that delivers a drug selected to alleviate pelvic pain to a genital nerve branch of at least one genitofemoral nerve of a patient, and an implantable electrical stimulation device that delivers electrical stimulation selected to alleviate pelvic pain to a genital nerve branch of at least one genitofemoral nerve of the patient.

In a further embodiment, the invention provides a method comprising delivering a drug to at least a portion of a genitofemoral nerve of a patient via an implanted drug delivery device.

In another embodiment, the invention provides an implantable drug delivery device that delivers a drug selected to alleviate pelvic pain to a genitofemoral nerve of a patient, and an implantable electrical stimulation device that delivers electrical stimulation selected to alleviate pelvic pain to a genital nerve branch of at least one genitofemoral nerve of the patient.

In various embodiments, the invention may provide one or more advantages. For example, delivering a drug to a genitofemoral nerve of a patient may substantially reduce or eliminate pelvic pain such as that caused by chronic testicular pain (CTP), post vasectomy pain, genitofemoral neuralgia, and other conditions that cause long term pain in the testicles, groin, or abdomen, as well as other forms of pelvic pain experienced by female patients.

Testicle removal and spermatic cord denervation procedures that sever nerves in or adjacent to the spermatic cord, i.e., a genital branch of the genitofemoral nerve, ilioinguinal nerve, or sympathetic nerves, often result in unwanted side effects including loss of sensation in the testicles and/or scrotum and loss of the cremasteric reflex which may cause fertility issues. Therapeutic nerve blocks typically only relieve pain temporarily. In contrast, delivery of a drug to one or both genital nerve branches, either directly or via the respective spermatic cords, may provide permanent or long-lived effective therapy for many patients with fewer or no unwanted side effects.

In addition, for male patients, the fluid transfer devices of a drug delivery device may be implanted proximate to the spermatic cord using well known surgical procedures for exposing the spermatic cord, e.g., inguinal incision as used for spermatic cord denervation or hernia repair, providing ease of deployment by experienced surgeons or other caregivers.

The details of one or more embodiments of the invention are set forth in the accompanying drawings and the description below. Other features, objects, and advantages of the invention will be apparent from the description and drawings, and from the claims.

### BRIEF DESCRIPTION OF DRAWINGS

FIG. 1 is a schematic diagram illustrating an example system that includes an implantable medical device for delivering a drug to a genital nerve branch of a patient for alleviation of pelvic pain from a front view of a male patient.

FIG. 2 is a schematic diagram further illustrating the example system of FIG. 1 from a side view of a male patient.

FIG. 3 is a block diagram illustrating an example implantable medical device for delivering a drug to the genital nerve branch of a patient.

FIG. 4 is a block diagram illustrating an example clinician programmer that allows a clinician to program drug delivery for a patient.

FIGS. 5A and 5B are schematic diagrams illustrating an example system that includes an implantable medical device for delivering electrical stimulation in combination with one or more drugs to a genital nerve branch of a patient for alleviation of pelvic pain from a front view of a male patient.

FIGS. 6A-6C are schematic diagrams illustrating an example cuff electrode useful in a combined drug delivery and electrical stimulation system.

FIG. 7 is a schematic diagram further illustrating the example system of FIGS. 5A and 5B with a different type of electrical stimulator from a side view of a male patient.

FIGS. 8A and 8B are schematic diagrams illustrating incorporation of fixation elements in an electrode lead or a fluid transfer device.

FIG. 9 is a schematic diagram further illustrating the example system of FIG. 7 with another different type of electrical stimulator from a side view of a male patient.

FIGS. 10A-10C are schematic diagrams illustrating an example leadless microstimulator suitable for use in the system of FIG. 10.

FIG 11 is a side cross-sectional view of a leadless electrical microstimulator implanted within the spermatic cord.

FIG. 12 is a schematic diagram illustrating implantation of a leadless microstimulator within the spermatic cord or tissue adjacent the spermatic cord.

FIG. 13 is a functional block diagram illustrating various components of the leadless microstimulator of FIG. 11.

FIG. 14 is a schematic diagram illustrating another configuration for the example system of FIG. 7.

FIG. 15 is a flow chart illustrating a technique for delivering a drug to a spermatic cord of a patient for alleviation of pelvic pain.

### DETAILED DESCRIPTION

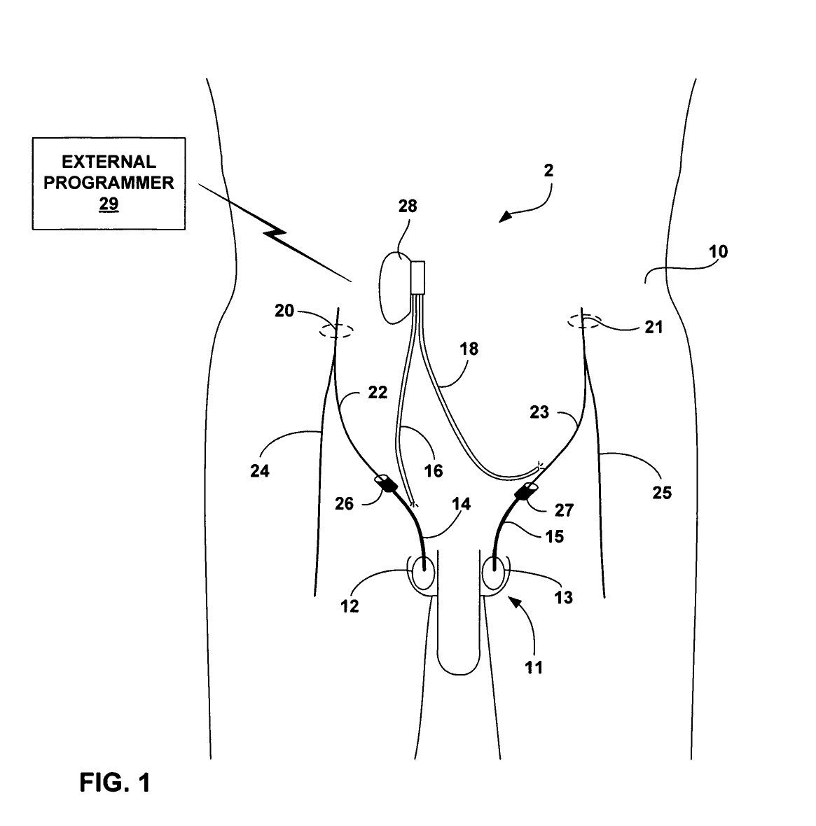

FIG. 1 is a schematic diagram illustrating an example system 2 that includes an implantable medical device (IMD) 28 in the form of a drug delivery device that delivers one or more drugs to one or both genitofemoral nerves or the genital branch of one or both genitofemoral nerves, either directly for a male or female patient, or via spermatic cords 14 and 15 of a male patient 10. In FIG. 1, system 2 is illustrated from a front view perspective of patient 10. Of course, delivery of a drug to spermatic cords 14, 15 applies only in the case of male patients. Although the invention may be generally applicable to treat pelvic pain in both men and women, application of the invention to men will be described throughout this disclosure for purposes of illustration. Throughout the figures accompanying this disclosure, various anatomical features of patient 12 and structural features of system 2 are illustrated conceptually for ease of illustration. Accordingly, the figures may not necessarily present appropriate scales and proportions of such anatomical features. Rather, the drawings are provided as a conceptual rendering of such features to aid in the understanding of pertinent embodiments of the invention.

In the example of FIG. 1, IMD 28 delivers a drug to patient 10 for alleviation of chronic testicular pain (CTP), post vasectomy pain, genitofemoral neuralgia, and other conditions that cause long term (chronic) pain in the testicles (in a male patient), groin, or abdomen. CTP may be caused by on-going inflammation of the testicle (orchitis) or epididymis (epdidymitis), trauma, tumors, hernia, torsion (twisting of the testicle), varicocele, hydrocele, spermatocele polyarteritis nodosa, and previous surgical interventions such as vasectomy and hernia surgery. As an example, CTP or genitofemoral neuralgia may be attributed to nerve injury, such as stretching of a nerve, electrocoagulation, stricture caused by ligation, entrapment of the nerve in scar tissue, or irritation because of proximity to a zone of inflammation, during inguinal herniorrhaphy. In particular, damage to the genitofemoral nerve and, more particularly, the genital branch of the genitofemoral nerve may cause a patient to experience pain in the testicles or associated scrotal area.

IMD 28 may also deliver one or more drugs to patient 10 for alleviation of chronic pelvic pain that is idiopathic in origin. Drug delivery parameters, such as which of a plurality of drugs contained in the device are delivered and the dosage and rate at which the drugs are delivered, may be selected as appropriate to alleviate pain for the particular patient 10. By way of example, and without limitation, the IMD 28 may deliver one or more of a variety of drugs, such as gabapentin, morphine, clonidine, tizanidine, hydromorphone, fentanyl, sufentanil, methadone, meperidine, tetracaine, bupivicaine, zinconotide, adenosine, ketorolac, baclofen, ropivicaine, ketamine, octreotide, neostigmine, and droperidol. In general, such a drug may be selected to alleviate pain or otherwise modulate nerve response to alleviate pain or other symptoms.

In additional embodiments, IMD 28 delivers one or more drugs to a female patient (not shown) for alleviation of pelvic pain such as urogenital pain and idiopathic pain. Examples of pain in female patients include pain resulting from surgical procedures, non-surgical procedures, vulvodynia, and interstitial cystitis (painful bladder syndrome). Nerve injury may be caused by various surgical procedures including urological operations in the pelvic area, gynecological surgery, and hysterectomy. Non-surgical conditions which cause pain in women include, for example, adhesions, endometriosis, and pelvic congestion. Delivering a drug to the genitofemoral nerve or genital branch of the genitofemoral nerve in accordance with selected parameters may alleviate pain experienced by female patients.

FIG. 1 illustrates genital branches 22, 23 and femoral branches 24, 25 of genitofemoral nerves 20, 21, respectively. Generally, for a male patient, IMD 28 may deliver one or more drugs to spermatic cords 14 and 15 via fluid transfer devices, e.g., catheters, conduit, or the like, coupled to IMD 28. The drugs are selected to block or attenuate pain signals from testicles 12 and 13 and the associated scrotal area 11 from reaching the central nervous system (CNS). As shown in the illustrated example of FIG. 1, the fluid transfer devices may be implanted proximate to various portions of the genital nerve branch or spermatic cord. However, the invention is not so limited. Rather, the invention also includes embodiments in which fluid transfer devices may be implanted proximate to genitofemoral nerves 20, 21, i.e., above the branch point of genital nerve branches 22, 23, respectively. In the illustrated example of FIG. 1, a dotted circle indicates an example drug delivery site along genitofemoral nerve 20, 21 above the respective branch point.

Further, the invention includes embodiments in which a fluid transfer device is implanted proximate to at least one of genitofemoral nerve 20, genitofemoral nerve 21, genital nerve branch 22, genital nerve branch 23, spermatic cord 14, and spermatic cord 15. For example, fluid transfer devices may be implanted proximate to genitofemoral nerve 20 and proximate to genital nerve branch 22. In another example, fluid transfer devices may be implanted proximate to genitofemoral nerve 20 and proximate to spermatic cord 14. In yet another example, fluid transfer devices may be implanted proximate to genital nerve branch 22 and proximate to spermatic cord 14. The invention further includes embodiments in which fluid transfer devices are implanted bi-laterally in any combination. Such embodiments are included without exhaustively listing all possible combinations. Accordingly, the positions of fluid transfer devices 16 and 18 in FIG. 1 are merely exemplary.

The pain experienced by the patient may be unilateral or bilateral, constant or intermittent, spontaneous or exacerbated by physical activities and pressure, and may remain localized or radiate outward. In a male patient, for example, testicular pain may remain localized in the scrotum or radiate to the groin, perineum, back, or legs. Delivering one or more drugs to the genital branch of a patient blocks or prevents pain signals from testicles 12 and 13 and associated scrotal region 11 from reaching the CNS based on the type of drug delivered and position of the fluid transfer devices. Accordingly, the drug or drugs contained in IMD 28 and the position of fluid transfer devices 16 and 18 are largely based on the pain perceived by patient 10.

In the illustrated example, IMD 28 is shown coupled to fluid transfer devices 16 and 18 that deliver drugs to spermatic cords 14 and 15, respectively. Fluid transfer devices 16 and 18 may comprise a catheter, a conduit, or the like, that enables the transfer of fluid from IMD 28 to the delivery site, i.e., spermatic cords 14 and 15. Fluid transfer devices 16 and 18 deliver a drug from a reservoir within IMD 28 to the target site, i.e., spermatic cords 14 and 15. IMD 28 may include one or more reservoirs. Each reservoir may contain a drug or a mixture of drugs. For example, as mentioned previously, a reservoir may contain any of a variety of drugs such as gabapentin, morphine, clonidine, tizanidine, hydromorphone, fentanyl, sufentanil, methadone, meperidine, tetracaine, bupivicaine, zinconotide, adenosine, ketorolac, baclofen, ropivicaine, ketamine, octreotide, neostigmine, or droperidol. In some embodiments, each fluid transfer device may be coupled to the same reservoir or different reservoirs. IMD 28 also may include one or more pumps that deliver drugs from the reservoirs to the fluid transfer devices.

A reservoir within IMD 28 may comprise a self-sealing reservoir that may be refilled by a needle and syringe, and need not be surgically removed when empty. The needle and syringe may also be used to drain a pump of one drug, flush the reservoir, and refill the reservoir with a different drug. Examples of such implantable IMDs include a number of SynchroMed™ pumps manufactured by and commercially available from Medtronic Inc. of Minneapolis, Minnesota. The invention is not limited to use with Synchromed™ pumps, however, and may be adapted for use with other implantable drug delivery devices.

IMD 28 includes a processor that controls the delivery of drugs to spermatic cords 14 and 15. The processor may, for example, control which drugs are delivered by IMD 28 by controlling which pumps are active. The processor may also control the dosage and rate at which the drugs are delivered by IMD 28 by controlling the activity of the pumps. The processor may be programmed prior to implanting IMD 28 with patient or, alternatively, programmed via external programmer 29. A clinician may use external programmer 29 to program a drug delivery method for patient 10. For example, the drugs may be delivered by a constant drip, a periodic bolus, a combination of these methods, or another delivery method. The present invention is not limited to a particular drug delivery method.

Fluid transfer devices 16 and 18 may be implanted proximate to spermatic cords 14 and 15, respectively. In the illustrated example, fluid transfer device 16 is implanted proximate to spermatic cord 14 and fluid transfer device 18 is implanted proximate to the genital branch 23 of genitofemoral nerve 21, but the invention is not limited as such. Rather, fluid transfer device 16 may be implanted at various locations along spermatic cord 14, genital nerve branch 22, genitofemoral nerve 20, or sympathetic nerves (not shown). Spermatic cord 14 includes a lower portion of the genital nerve branch 22 of the genitofemoral nerve 20. Similarly, fluid transfer device 18 may be implanted at various locations along spermatic cord 15, genital nerve branch 23, genitofemoral nerve 21, or sympathetic nerves (not shown).

The positions of fluid transfer devices 16 and 18 in FIG. 1 are shown for purposes of illustration to show different possible implantation locations and associated target sites. Specifically, fluid transfer devices 16 and 18 illustrate two locations which may be particularly advantageous for delivering a drug, which will be described in detail below. However, IMD 28 may be coupled to a single fluid transfer device or a plurality of fluid transfer devices based on the perceived pain of the patient and his response to drug delivery therapy.

In FIG. 1, spermatic cords 14 and 15, genitofemoral nerves 20 and 21, and genital branches 22, 23 and femoral branches 24, 25 of genitofemoral nerves 20 and 21 are illustrated. FIG. 1 also illustrates inguinal canals 26 and 27. The genitofemoral nerve 20, 21 originates from the L1 and L2 nerves in the lumbar region (lower back) at L1/L2. As the genitofemoral nerve 20, 21 passes through the lumbar region, it crosses behind the ureter (not shown). Slightly posterior to and at a variable distance above the inguinal ligament (not shown), the genitofemoral nerve 20, 21 divides into the genital branches 22, 23 and femoral branches 24, 25. The genital branches 22, 23 cross the transverses abdominus (not shown) and internal oblique muscles (not shown) and enter the respective inguinal canal 26, 27 through the internal inguinal ring.

Within the inguinal canal 26, 27, the genital branch 22, 23 runs along the spermatic cord 14, 15, respectively. The spermatic cord includes various layers (not shown). These layers are the external spermatic fascia, cremasteric muscle and fascia, genitofemoral nerve, internal spermatic fascia, ductus deferens, lymph vessels, pampiniform plexus of veins which become the testicular vein, and testicular artery. More specifically, as the structures within the spermatic cord pass through the transversalis fascia (not shown), they join with one of the layers of the spermatic cord, the internal spermatic fascia.

As the spermatic cord 14, 15 continues through the inguinal canal, in a male patient, it joins with the cremasteric layer of muscle and fascia from the internal oblique muscle. These muscle fibers perform an important reflex, i.e., the cremasteric reflex. When the cremasteric muscle contracts, the testicle is pulled closer to the body. This reflex keeps the testicles at the correct temperature, for example, by relaxing when the testicles are too warm and contracting when the testicles are too cold. If the cremasteric reflex is absent or functions incorrectly, e.g., due to denervation or resection, the male may experience fertility related issues.

Finally, when the spermatic cord 14, 15 passes through the superficial ring, it joins an external spermatic fascia layer derived from the aponeurosis of the external oblique. After the spermatic cord 14, 15 traverses the inquinal canal 26, 27, it extends into the scrotum and to the testes 12, 13 where the genital branch 22, 23 of the genitofemoral nerve innervates the testes 12, 13. Drugs may be delivered via fluid transfer devices positioned proximate to the spermatic cord superior to a testicle and inferior to an inguinal canal of the patient, or positioned proximate to a genital branch of the genitofemoral nerve superior to an inguinal canal and inferior to the genitofemoral nerve of the patient.

In the illustrated example, fluid transfer device 16 is implanted proximate to the external fascia of spermatic cord 14 and connected to IMD 28. Fluid transfer device 16 may include fixation elements for securing fluid transfer device 16 to spermatic cord 14 or tissue adjacent to spermatic cord 14. Fixation elements may improve the targeting of the drug delivered by fluid transfer device 16 to spermatic cord 14. Fluid transfer device 16 delivers the drug to the genital branch 22 of genitofemoral nerve 20 through the fascia. The fascia protects genital nerve branch 22 within spermatic cord 14 from being in direct contact with the fluid transfer device. Direct contract may be undesirable because the nerve may become damaged by the fixation elements as the patient moves or if the fluid transfer device is removed. In addition, delivering the drug to the spermatic cord may result in the patient experiencing more complete or prolonged relief from pain and fewer unwanted side effects because the drug affects a larger portion of tissue and nerves within the spermatic cord.

Fluid transfer device 18, in the illustrated example, is implanted proximate to genital nerve branch 23 above inguinal canal 27 and below genitofemoral nerve 21. Because fluid transfer device 18 is located higher (upstream in the central nervous system) than fluid transfer device 16, patient 10 may experience relief from pain over a larger area, which may be advantageous in some instances. However, genital nerve branch 23 does not include an external fascia to serve as a protective layer in this region. Consequently, implanting fluid transfer device 18 proximate to genital nerve branch 23 may inherently have a greater risk of damaging the nerve, possibly reducing the long-term efficacy of the drug therapy. As a result, additional care may be necessary when implanting a fluid transfer device proximate to the genital nerve branch above the inguinal canal and below the genitofemoral nerve, as shown with respect to fluid transfer device 18.

The positions of fluid transfer devices 16, 18 in FIG. 1 are for purposes of illustration of different possible positions. In practice, one or both fluid transfer devices 16, 18 may be positioned above inguinal canal 27 and below genitofemoral nerve 21 to directly stimulate genital nerve branch 23. Alternatively, one or both fluid transfer devices 16, 18 may be positioned below inguinal canal 27 and above testes 12, 13 to indirectly stimulate genital nerve branch 23 indirectly via spermatic cord 15. As discussed previously, fluid transfer devices may be positioned based on the pain perceived by the patient and the type of electrical stimulation delivered to treat the pain. In general, fluid transfer devices may be implanted proximate to the spermatic cord above or below the inguinal canal to deliver drugs to the genital branch of the genitofemoral nerve to treat pelvic pain such as CTP. Alternatively, fluid transfer devices may be implanted proximate to the genitofemoral nerve to deliver drugs for the treatment of pelvic pain such as CTP.

In general, to treat pelvic pain such as CTP, fluid transfer devices may be implanted proximate to the spermatic cord above or below the inguinal canal to deliver drugs to the genital branch of the genitofemoral nerve or proximate to the genitofemoral nerve to deliver drugs to the genitofemoral nerve. It may be difficult to implant a fluid transfer device proximate to the spermatic cord within the inguinal canal because of the dense gathering of tissue in this region. Consequently, implanting a fluid transfer device proximate to the spermatic cord within the inguinal canal may result in unwanted damage to tissue, muscle, blood vessels and/or nerves within the inguinal canal, such as the cremasteric muscle and blood vessels supplying blood to the testicles, which may cause fertility issues or even loss of blood flow causing the testicle to die.

Fluid transfer devices 16 and 18 are typically either surgically implanted or inserted percutaneously. Fluid transfer devices 16 and 18 may be surgically implanted using well known surgical techniques. For example, the surgical procedure for exposing the spermatic cord is well defined, i.e., inguinal incision as used for spermatic cord denervation or hernia repair. A surgical procedure for genitofemoral neurectomy is described in detail in Judith A. Murovic et. al, “Surgical Management of 10 Genitofemoral Neuralgias at the Louisiana State University Health Sciences Center,” Neurosurgery, Volume 56, Number 2, pages 298-303, February 2005. A procedure for spermatic cord denervation is described in detail in Laurence A. Levine et al., Microsurgical Denervation of the Spermatic Cord as Primary Surgical Treatment of Chronic Orchialgia, The Journal of Urology, Vol. 165, pages 1927-1929, June 2001.

Prior to surgically implanting fluid transfer devices, local nerve blocks may be performed using a nerve blocking agent to determine the precise nerve involved in the pain experienced by the patient. For example, if a spermatic nerve block ameliorates the patient's pain, a surgeon may conclude that drug therapy as described herein is likely to be efficacious, and may proceed to surgically implant fluid transfer devices in accordance with the invention. Alternatively, a clinician may stimulate the patient using an insulated needle to determine the nerve involved and the placement of a fluid transfer device. The diagnosis may also be made using the results of the patient history, physical examination, and preoperative electromyography.

IMD 28 may be implanted at a site in patient 10 near spermatic cords 14 and 15. The implantation site may be a subcutaneous location in the side of the lower abdomen. Alternatively, IMD 28 may be implanted within the scrotum of the patient. In this case, IMD 28 may be miniaturized to allow IMD 28 to be implanted within the scrotum. In any case, the surgeon may then tunnel a fluid transfer device through tissue and subsequently connect the fluid transfer device to IMD 28. IMD 28 may be constructed with a biocompatible housing, such as titanium or stainless steel, much like a conventional implantable drug pump such as those used for spinal cord, deep brain, and cardiac drug delivery.

External programmer 29 may control drug delivery by IMD 28. For example, in some embodiments, external programmer 29 may comprise a clinician programmer or a patient programmer. A clinician programmer may be a handheld computing device including a display, such as an LCD or LED display, to display drug delivery parameters. A clinician programmer may also include a keypad, which may be used by a user to interact with the clinician programmer. In some embodiments, the display may be a touch screen display, and a user may interact with the clinician programmer via the display. A user may also interact with the clinician programmer using peripheral pointing devices, such as a stylus, mouse, trackball, scroll wheel or the like. The keypad may take the form of an alphanumeric keypad or a reduced set of keys associated with particular functions.

A clinician (not shown) may use the clinician programmer to program drug therapy for patient 10. In particular, the clinician may use the clinician programmer to select values for therapy parameters, such as the dosage and rate at which the drug is delivered, for one of or both fluid transfer devices 16 and 18. IMD 28 may deliver the drugs according to programs, each program including values for a plurality of such therapy parameters. The therapy parameters also may specify particular drugs to be delivered at different times, in the event IMD 28 is equipped for delivery of multiple drugs. IMD 28 controls delivery of drug therapy according to preprogrammed programs and schedules.

When implemented as a patient programmer, external programmer 29 may be a handheld computing device. The patient programmer 26 may also include a display and a keypad to allow patient 10 to interact with the patient programmer. In some embodiments, the display may be a touch screen display, and patient 10 may interact with the patient programmer via the display. Patient 10 may also interact with the patient programmer using peripheral pointing devices, such as a stylus or mouse.

Patient 10 may use the patient programmer to control the delivery of drug therapy. In particular, in response to a command from patient 10, external programmer 29 may activate IMD 28 to deliver drugs or, alternatively, deactivate IMD 28 when no drugs are desired. Patient programmer 26, IMD 28, or both may apply maximum dosage and rate limits, and lockout intervals, to prevent delivery of excessive amounts of the drug in response to patient requests. Patient 10 may also use the patient programmer to select the programs that will be used by IMD 28 to deliver the drugs. Further, patient 10 may use the patient programmer to make adjustments to programs, such as adjustments to which of a plurality of drugs are delivered and the dosage and rate at which the drugs delivered. Additionally, the clinician or patient 10 may use a clinician or patient programmer to create or adjust schedules for delivery of drugs.

IMD 28 and external programmer 29, implemented as a clinician programmer or a patient programmer, communicate via wireless communication. In particular, external programmer 29 communicates via wireless communication with IMD 28 using radio frequency (RF) telemetry techniques known in the art. The clinician programmer and patient programmer may communicate with one another by wireless communication, e.g., to change or update programs. Alternatively, the programmers may communicate via a wired connection, such as via a serial communication cable, or via exchange of removable media, such as magnetic or optical disks, or memory cards.

As previously described, fluid transfer devices 16 and 18 may be implanted surgically or percutaneously. When inserted percutaneously, fluid transfer devices 16 and 18 may be used in conjunction with an external drug delivery device (not shown) in order to determine if permanent implantation of the fluid transfer devices is an effective treatment for the patient's pain. For example, prior to implantation of IMD 28, patient 10 may engage in a trial period, in which patient 10 receives an external drug delivery device on a temporary basis. The external drug delivery device is coupled to percutaneous fluid transfer devices The external drug delivery device may be coupled to one or more temporary fluid transfer devices or chronically implanted fluid transfer devices via a percutaneous catheter extension.

The trial drug delivery device permits a clinician to observe drug therapy efficacy and determine whether implantation of a chronic drug delivery device is advisable. Specifically, the trial drug delivery device period may assist the clinician in selecting values for a number of programmable parameters in order to define the drug therapy delivered to patient 10. For example, the clinician may select a one or more particular drugs or a mixture of drugs to be delivered to patient 10, as well as the dosage and rate at which the drugs delivered. If chronic implantation is indicated, the physician may withdraw the percutaneous fluid transfer device or devices. Alternatively, the percutaneous fluid transfer devices may be designed for chronic implantation, in which case they can be disconnected from an external drug delivery device and coupled to an implanted drug delivery device.

By delivering drugs to nerves associated with the spermatic cord, a system in accordance with an embodiment of the invention may substantially reduce or eliminate pelvic pain such as CTP, post vasectomy pain, genitofemoral neuralgia, and other conditions that cause long term pain in the testicles, groin, or abdomen. Testicle removal and spermatic cord denervation procedures may result in permanent and substantial pain relief but may also cause unwanted side effects, such as loss of sensation in the testicle and/or scrotum, loss of the cremasteric reflex which may cause fertility issues, and even loss of blood flow causing the testicle to die. Therapeutic nerve blocks may also be used to treat CTP, but generally only relieve pain temporarily. Because delivering drugs to a spermatic cord via an implantable drug delivery device does not require severing any nerves associated with the spermatic cord and, more particularly, aims to avoid damaging nerves, the invention may provide similar or improved pain relief without the unwanted side effects.

The invention is not limited to delivering drug therapy to treat CTP and other conditions that cause long term pain in the pelvic or groin region. Rather, the invention also may include embodiments in which electrical stimulation is delivered in combination with drug therapy to one or both genital nerve branches, directly or indirectly via the spermatic cord. Electrical stimulation and drug therapy may be delivered simultaneously or on an alternating basis. For example, drug therapy may be delivered constantly or intermittently through the course of a day and the patient may use a patient programmer to deliver electrical stimulation when experiencing moments of increased pain. Alternatively, electrical stimulation may be delivered according to preprogrammed parameter sets and schedules and the patient may use a patient programmer to deliver drug therapy when the electrical stimulation does not substantially reduce the pain. In either case, the combined delivery of electrical stimulation and one or more druges supports neuromodulation therapy to alleviate pain or other symptoms associated with pelvic region disorders.

In some embodiments, system 2 includes an implantable stimulation device that applies electrical stimulation to the genital branch of one or both genitofemoral nerves or one or both genitofemoral nerves in combination with the previously described drug therapy. Such systems include one or more electrical stimulators that apply electrical stimulation to the genitofemoral nerve of the genital branch of the genitofemoral nerve, e.g., directly or via the spermatic cord in a male patient, to alleviate CTP or other afflictions associated with pelvic pain in men and women.

The electrical stimulators may comprise various types of electrodes such as cuff electrodes, electrode leads, and/or microstimulators implanted at various locations proximate to one or both of the genitofemoral nerves of a patient or the genital branch of one or both genitofemoral nerves to apply stimulation uni-laterally or bi-laterally. As an example, electrode leads (not shown) may each include a cuff electrode (not shown) that delivers electrical stimulation therapy to spermatic cords 14 and 15, respectively.

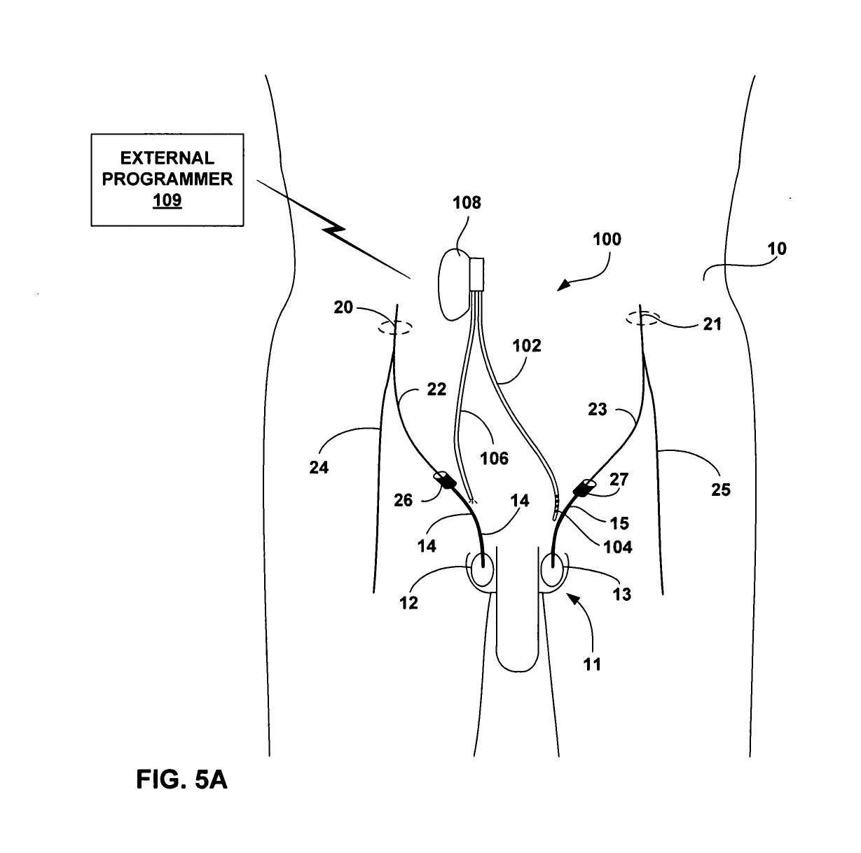

FIG. 5B illustrates an example system in which an IMD is coupled to a cuff electrode that stimulates a genital nerve branch of a patient, either directly or via a spermatic cord and a fluid transfer device that delivers a drug to the other genital nerve branch of the patient as previously described. A cuff electrode includes a cuff-like fixation structure and one or more electrodes carried by the fixation structure. Cuff electrodes may be implanted at different locations along spermatic cords 14 and 15, respectively. As a result, patient 10 may experience parasthesia in different areas on each side of his body in response to electrical stimulation delivered by the cuff electrodes.

In particular, a cuff electrode may be wrapped around the external fascia of a spermatic cord and connected to the implantable stimulation device via a lead 18 and, optionally, a lead extension. The electrical stimulation applied by the cuff electrode stimulates the genital nerve branch through the fascia. The fascia protects the genital nerve branch within the spermatic cord from being in direct contact with the cuff electrode, thus avoiding adhesion. Adhesion may be undesirable because the nerve may become damaged as the patient moves or if the electrode is removed. Thus, it may be desirable to deliver electrical stimulation to the spermatic cord by wrapping a cuff electrode around the spermatic cord below the inguinal canal and above the attached testicle.

Cuff electrodes may comprise a rigid cuff electrode, a self-sizing spiral cuff electrode, a half cuff electrode, a helical electrode, a chambered electrode, or other types of cuff electrodes that are shaped, sized and otherwise configured to at least partially wrap around a spermatic cord. The cuff electrode may be sized and shaped to at least partially enclose the spermatic cord and promote electrical coupling pressure between the electrode and the fascia of the spermatic cord. Cuff electrodes may include a single electrode or multiple electrodes. For example, a cuff electrode may include a bipolar or multipolar arrangement of electrodes or a unipolar electrode that is referenced to the electrical potential of an active can electrode carried by, for example, IMD 28.

The invention is not limited to embodiments in which IMD 28 or an independent implantable stimulation device is coupled to cuff electrodes. Instead, IMD 28 may be coupled to any number and any type of electrodes, such as conventional ring electrode leads, paddle electrode leads, and other electrodes suitable for delivering electrical stimulation to the spermatic cord. In addition, in some cases, leadless stimulators may be used. Further, the invention is not limited to embodiments that deliver electrical stimulation to a specific area of the spermatic cord.

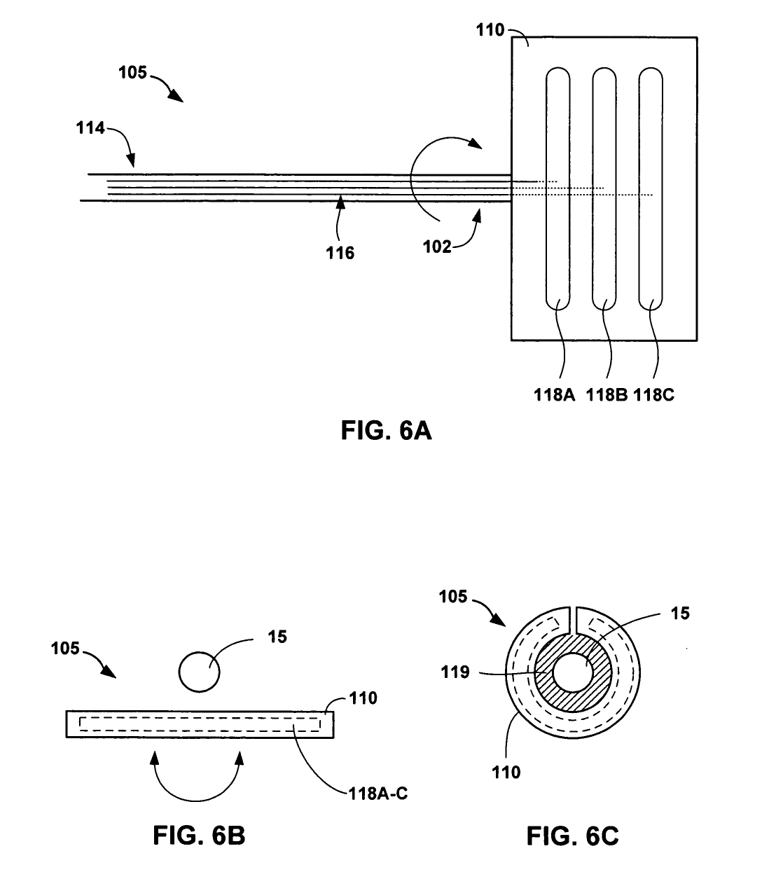

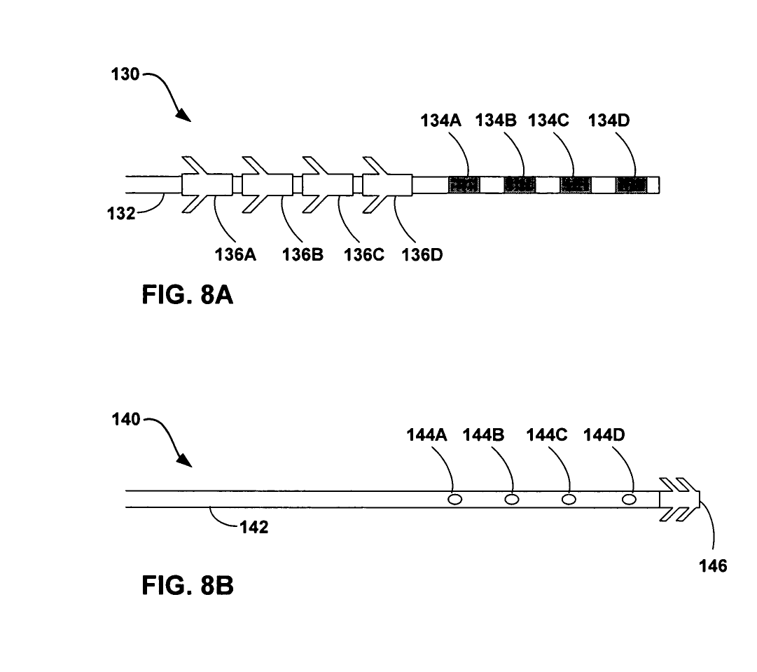

As an example, FIG. 7 illustrates another example system in which an IMD is coupled to an electrode lead having electrodes displaced on the distal end of the lead to stimulate a genital nerve branch of a patient, either directly or via a spermatic cord. As another example, FIG. 9 illustrates a leadless microstimulator implanted within the fascia of a spermatic cord. In this case, an IMD or external programmer may wirelessly control the leadless microstimulator to deliver electrical stimulation to the fascia of the spermatic cord. In addition, although not illustrated, an IMD may also be coupled to an electrode suitable for applying electrical stimulation to the genitofemoral nerve.

The electrical stimulators may be coupled to an implantable stimulation device implanted within a subcutaneous pocket in the abdomen of the patient or, alternatively, the scrotum of the patient. The implantable stimulation device may be incorporated within IMD 28 or may be independent of IMD 28. In any case, the electrical stimulators may be coupled to the stimulation device via standard implantable electrode leads. Alternatively, leadless microstimulators may be capable of wireless communication with IMD 28, external programmer 29, or both.

The implantable stimulation device includes electrical stimulation pulse generator circuitry and delivers electrical stimulation in the form of electrical pulses in accordance with stored stimulation parameters, e.g., electrode polarity, pulse amplitudes, pulse widths, and pulse rates. By way of example, the electrical stimulation may include stimulation pulses having pulse widths between approximately 10 and 5000 microseconds, more preferably between approximately 100 and 1000 microseconds and still more preferably between 180 and 450 microseconds. The stimulation pulses may define voltage amplitudes between approximately 0.1 and 50 volts, more preferably between approximately 0.5 and 20 volts and still more preferably between approximately 1 and 10 volts. The pulses may define frequencies between approximately 0.5 and 500 hertz, more preferably between approximately 10 and 250 hertz and still more preferably between approximately 50 and 150 hertz. The pulses may be alternating current (ac) pulses or direct current (dc) pulses, and may be mono-phasic, bi-phasic, or multi-phasic in various embodiments.

The implantable stimulation device may drive each of the electrodes with the same or different stimulation pulses or waveforms. In some embodiments, the implantable stimulation device may cause each of the electrodes to deliver electrical stimulation simultaneously, or in an interleaved or alternating fashion. For example, each of the electrodes may deliver electrical stimulation with different pulse rates, duty cycles or scheduled times for delivery, which may result in alternating delivery of stimulation. Interleaved or alternating delivery of stimulation may, for example, reduce the likelihood that neural accommodation or tolerance will impair the efficacy of the stimulation. Interleaved or alternating delivery of stimulation may also result in more complete pain relief than would be possible through delivery of stimulation via only one electrode or electrode array. Interleaved stimulation may be delivered via any combination of ring electrodes, paddle lead electrodes, cuff electrodes, or microstimulators.

In addition to programming drug therapy for patient 10, a clinician or patient 10 may also use external programmer 29 to program electrical stimulation delivered to patient 10. In particular, the clinician may use the clinician programmer to select values for therapy parameters, such as pulse amplitude, pulse width, pulse rate, electrode polarity and duty cycle, for each of the electrodes coupled to the implantable stimulation device. The implantable stimulation device may deliver the electrical stimulation according to programs, each program including values for a plurality of such therapy parameters. Patient 10 may use the patient programmer to control the delivery of electrical stimulation. In particular, in response to a command from patient 10, external programmer 29 may activate the implantable stimulation device to deliver electrical stimulation or, alternatively, deactivate the implantable stimulation device when no electrical stimulation is desired. Patient 10 may also use the patient programmer to select the programs that will be used by the implantable stimulation device to deliver electrical stimulation. Further, patient 10 may use the patient programmer to make adjustments to programs, such as adjustments to amplitude, pulse width and/or pulse rate. Additionally, the clinician or patient 10 may use a clinician or patient programmer to create or adjust schedules for delivery of electrical stimulation.

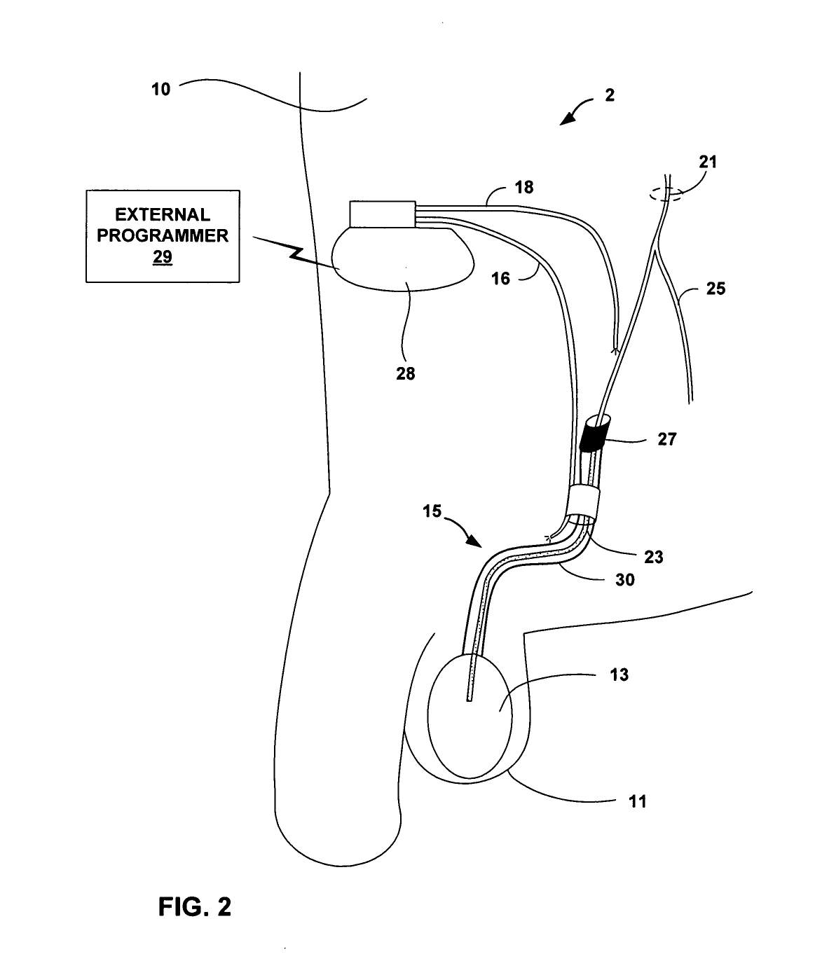

FIG. 2 is a schematic diagram further illustrating system 2. In particular, system 2 is illustrated from the left side of patient 10. For purposes of illustration, only spermatic cord 15, genital nerve branch 23, femoral nerve branch 25, genitofemoral nerve 21, and testicle 13 are shown. Furthermore, fluid transfer device 16 is illustrated as being implanted proximate to spermatic cord 15 to illustrate the different locations at which fluid transfer devices may be implanted and to illustrate an embodiment in which multiple fluid transfer devices are implanted along a single spermatic cord 15. Accordingly, fluid transfer device 18 is shown as being implanted proximate to genital nerve branch 23, while fluid transfer device 16 is shown as being implanted proximate to spermatic cord 15. Following the convention illustrated in FIG. 1, a dotted circle illustrates an example stimulation site at which a fluid transfer device may be implanted proximate to genitofemoral nerve 21 in combination with one or more of fluid transfer devices 16 and 18. In an embodiment in which two or more fluid transfer devices are implanted along the same spermatic cord, the fluid transfer devices may be coupled to the same or different reservoirs within IMD 28 and, accordingly, deliver the same or different drugs to patient 10.

FIG. 2 illustrates genital nerve branch 23 originating from genitofemoral nerve 21 and passing through inguinal canal 27 to innervate testicle 13. As previously described, spermatic cord 15 joins an external fascia layer 30 as it passes through the superficial ring of the inguinal canal. Genital nerve branch 23 is shown within the external fascia 30 of spermatic cord 15. Fluid transfer device 16 is implanted proximate to external fascia 30 of spermatic cord 15. External fascia 30 may serve to protect genital nerve branch from being damaged when fluid transfer device 16 is implanted. In particular, external fascia 30 prevents fluid transfer device 16 from being in direct contact with genital nerve branch 23 which may prevent damage to genital nerve branch 23. When implanted proximate to spermatic cord 15, fluid transfer device 16 delivers the drug indirectly to genital nerve branch 23 via external fascia 30 of spermatic cord 15. Delivering the drug to spermatic cord 15 may result in a more complete or prolonged relief from pain as well as fewer unwanted side effects because the drug may disperse or diffuse over a larger portion of external fascia 30 and nerves within spermatic cord 15.

In general, fluid transfer devices 16 and 18 may include fixation means such as sutures or anchoring mechanisms that enable fluid transfer devices 16 and 18 to remain in place as patient 10 moves. Such fixation means may damage tissue or the nerve itself, possibly causing additional pain which may reduce the efficacy of the drug therapy. Consequently, fluid transfer devices 16 and 18 may be implanted proximate to spermatic cord 15 and genital nerve branch 23 by fixing fluid transfer devices 16 and 18 to tissue adjacent to spermatic cord 15 and genital nerve branch 23 via fixation means.

In other embodiments, however, fluid transfer devices may include a fixation structure, e.g., similar to the cuff of a cuff electrode, that at least partially wraps around spermatic cord 15 and genital nerve branch, respectively. The fixation structure may be fabricated from a flexible biocompatible material that provides a flexible interface between the fluid transfer device and the tissue, i.e., spermatic cord 15 or genital nerve branch 23. In such cases, the fixation structure may form a split cylinder or a “U” shape sized to fit around the spermatic cord or genital nerve branch. When implemented as cuff style fluid transfer device, fluid transfer devices 16 and 18 may generally comprise a rigid cuff fluid transfer device, a self-sizing spiral cuff fluid transfer device, a half cuff fluid transfer device, a helical fluid transfer device, a chambered fluid transfer device, and other types of cuff fluid transfer devices that at least partially wrap around a spermatic cord. Upon enclosure of at least a portion of the spermatic cord, a cuff may be held in a closed position by shape memory properties, sutures, interlocking tabs, surgical adhesive, crimping, or other fixation techniques or structures. For reference, FIGS. 7A-C illustrate example cuff electrodes that may be useful in delivering electrical stimulation in combination with the described drug therapy and, more particularly, the fixation structure of such cuff electrodes.

Fluid transfer devices 16 and 18 may also, in some embodiments, not include any form of fixation means. In such embodiments, fluid transfer devices 16 and 18 may move relative to spermatic cord 15 and genital nerve branch, but remain within an acceptable region associated with the target delivery site for delivering drug therapy.

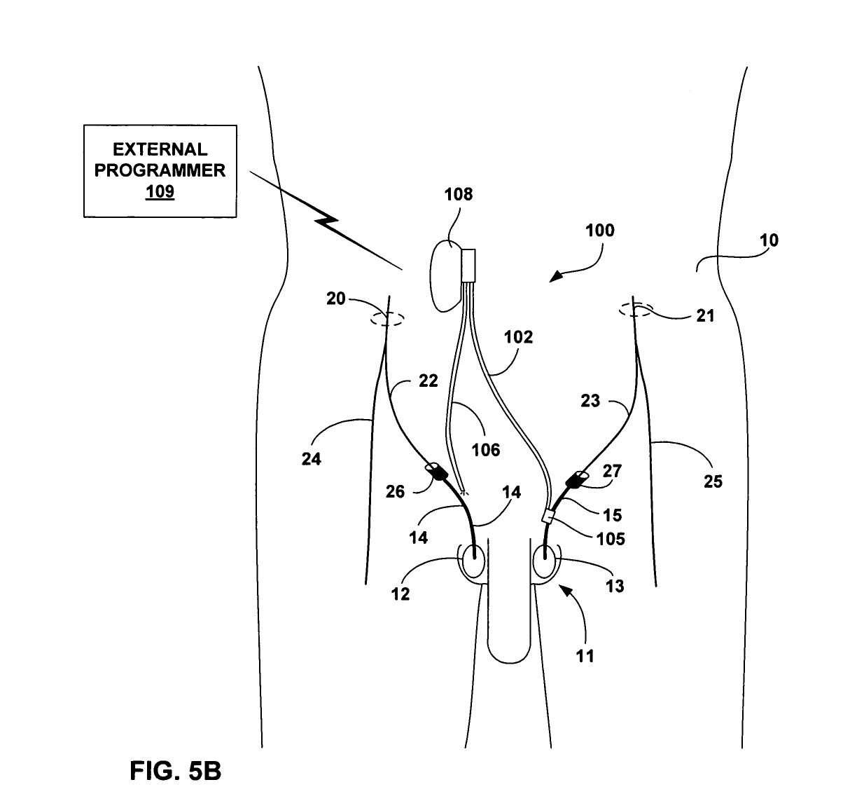

Again, system 2 may also include an implantable stimulation device that applies electrical stimulation to genital nerve branch 23 directly or indirectly via spermatic cord 15 in combination with drug therapy. For example, FIG. 6 illustrates an example system that includes an IMD for delivering electrical stimulation in combination with drug therapy to a genital nerve of a patient. Such systems include one or more electrical stimulators that apply electrical stimulation to alleviate CTP or other afflictions associated with pelvic pain in men and women. The electrical stimulators may comprise various types of electrodes such as cuff electrodes, electrode leads, and/or microstimulators.

Cuff electrodes provide may be fabricated similar to and provide the same advantageous previously described with respect to fluid transfer devices having a similar cuff-like fixation structure. In other words, cuff electrodes may be constructed in the same manner and of the same materials as described with respect to fluid transfer devices and wrap at least partially around a spermatic cord or genital nerve branch. In particular, with respect to the spermatic cord, a cuff electrode prevents the electrode from being in direct contact with the genital nerve branch which may result in a more pleasant paresthesia because electrical stimulation is delivered to the genital nerve branch indirectly. Additionally, the external fascia of the spermatic cord may provide a buffer that reduces the damage to the genital nerve branch when the patient moves.

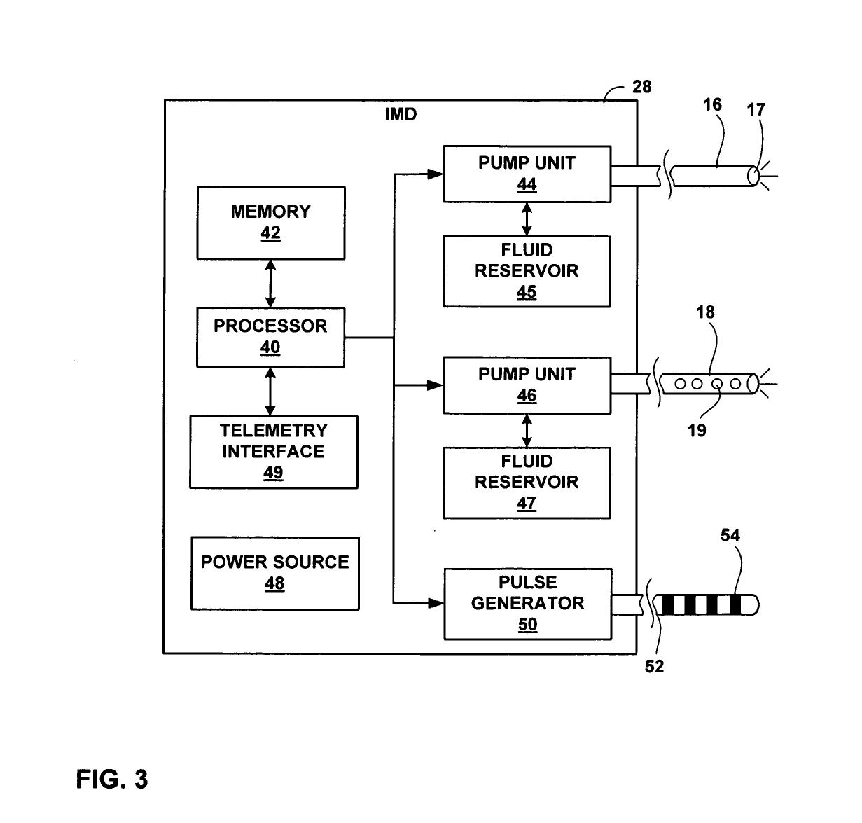

FIG. 3 is a block diagram illustrating an example configuration of IMD 28. IMD 28 may deliver one or more drugs to spermatic cord 15 and genital branch 23 of the genitofemoral nerve 21 of patient 10 via fluid transfer devices 16 and 18 as shown in FIG. 2. In some embodiments, however, an electrical stimulation device may also deliver electrical stimulation in combination with drug therapy to genital branch 23 directly or indirectly, via spermatic cord 15, via one or more electrical stimulators. In embodiments in which electrical stimulation is delivered to a genital nerve branch of a patient in combination with drug therapy, the electrical stimulation device may be incorporated with the drug delivery device or the electrical stimulation device and drug delivery device may be independent of each other, i.e., contained within separate housings. In the illustrated example of FIG. 3, IMD 28 incorporates the electrical stimulation device with the drug delivery device in a common housing.

By incorporating the drug delivery device and electrical stimulation device in a common housing of an IMD, circuitry associated with both devices, such as a processor and memory, may be shared and fabricated on a single circuit board. As a result, the IMD may be substantially smaller in size and cost less than separate drug delivery and electrical stimulation devices. Additionally, the IMD may be implanted within the patient using fewer incisions and requiring less space than separately implanting drug delivery and electrical stimulation devices.

In FIG. 3, IMD 28 is illustrated having fluid transfer devices 16 and 18 for delivering drug therapy and one or more electrodes 54, carried by one or more implantable leads 52, for delivering electrical stimulation to a patient. The configuration, type, and number of fluid transfer devices and electrodes in FIG. 3 are merely exemplary. In addition to, or in place of ring electrodes 54, IMD 28 may include any number and any type of electrodes, such as cuff electrodes, paddle electrode leads, and leadless stimulators. A leadless stimulator does not generally include any elongated leads, and instead carries electrodes on a housing of the stimulator or on a structure such as a fixation device extending from the housing.

Each fluid transfer device, e.g., a catheter, may have an elongated, tubular body with an inner lumen. With reference to FIG. 3, the body may include a proximal opening to receive the drug, and a distal opening 17 for delivery of the drug to a target site. Additionally, or alternatively, the elongated body may include a series of lateral outlets 19 formed in a lateral wall of the body. The outlets provide fluid communication between the inner lumen and the outside of the elongated body. The outlets 19 may be positioned at various axial positions along the length of the elongated body, as well as at various circumferential positions. The lateral outlets may be concentrated toward a distal end of the fluid transfer device.

In the example of FIG. 3, IMD 28 delivers one or more drugs to one or both genital nerve branches of a patient via fluid transfer devices 16 and 18 to alleviate CTP or other afflictions associated with pelvic pain in men and women. Fluid transfer devices 16, 18 may be coupled to a common fluid reservoir and pump unit, or separate fluid reservoirs 45, 47 and pump units 44, 46. IMD 28 may also apply electrical stimulation to one or both genital nerve branches of the patient via electrodes 54 in combination with the drug therapy. IMD 28 includes a processor 40, which may take the form of one or more microprocessors, digital signal processors (DSPs), application specific integrated circuits (ASICs), field-programmable gate arrays (FPGAs), other discrete or integrated logic circuitry, or any combination of such components. IMD 28 also includes pump unit 44, pump unit 46, and pulse generator 50 which operate under the control of processor 40 to deliver drugs and electrical stimulation to the patient.

In the example of FIG. 3, fluid transfer devices 16 and 18 are coupled to fluid reservoirs 45 and 47 via pump units 44 and 46, respectively. In some embodiments of the present invention, each fluid transfer device may be coupled to more than one reservoir, or more than one fluid transfer device may be coupled to a common reservoir. If fluid transfer devices 16, 18 are coupled to the same reservoir and pump unit, each fluid transfer device may simultaneously deliver the drug to respective target sites. Alternatively, if fluid transfer devices 16, 18 are intended to deliver the drug at different times, separate pump units or a valve coupled to a common pump unit may be provided to control flow to the fluid transfer devices.

Each of fluid reservoirs 45 and 47 may contain a drug or a mixture of drugs such as, gabapentin, morphine, clonidine, tizanidine, hydromorphone, fentanyl, sufentanil, methadone, meperidine, tetracaine, bupivicaine, zinconotide, adenosine, ketorolac, baclofen, ropivicaine, ketamine, octreotide, neostigmine, and droperidol. Pump units 44 and 46 pump the drugs from fluid reservoirs 45 and 47 to the target site via fluid transfer devices 16 and 18, respectively. Fluid reservoirs 45 and 47 may provide access for filling, e.g., by percutaneous injection of fluid via a self-sealing injection port. Fluid transfer devices 16 and 18 may comprise, for example, catheters that deliver, i.e., infuse or disperse, drugs from fluid reservoirs 45 and 47 to the same or different target sites along a genital nerve branch.

The target site may depend on the drug being delivered. Each of fluid transfer devices 16 and 18 may dispense drugs at one or more target sties. For example, one or both of fluid transfer devices 16 and 18 may deliver drugs directly to a genital nerve branch of a genitofemoral nerve, indirectly to a genital nerve branch via the spermatic cord, or to a genitofemoral nerve before the branch point. In some embodiments, fluid transfer devices 16 and 18 need not deliver drugs to the same target site.

Processor 40 controls delivery of drug therapy according to a selected parameter set stored in memory 56. Specifically, processor 40 may control pump units 44 and 46 to deliver drug therapy with a drug contained in IMD 28 and the dosage of the drug specified by the programs of the selected parameter set. For example, processor 40 may control which drugs are delivered by IMD 28 by controlling which of pump units 44 and 46 are active. Processor 40 may also control the dosage of the drugs delivered by IMD 28 by controlling the activity of pump units 44 and 46. Processor 40 may control each of pump units 44 and 46 to deliver drug therapy according to a different program of the parameter set. The drugs may be delivered by a constant drip, a periodic bolus, a combination of these methods, or some other delivery method. The invention is not limited to a particular drug delivery method.

Processor 40 may also control pulse generator circuit 50 to deliver electrical stimulation pulses with the amplitudes and widths, and at the rates specified by the programs of the selected parameter set. Processor 40 may also control pulse generator circuit 50 to deliver each pulse according to a different program of the parameter set.

Memory 42 may store parameter sets that are available to be selected by patient 10 for delivery of drug therapy and, in some embodiments, electrical stimulation. Memory 42 may also store schedules. Memory 42 may include any combination of volatile, non-volatile, removable, magnetic, optical, or solid state media, such as read-only memory (ROM), random access memory (RAM), electronically-erasable programmable ROM (EEPROM), flash memory, or the like.

IMD 28 delivers stimulation according to preprogrammed stimulation parameters and, optionally, schedules stored in memory 42. Schedules may define times for processor 40 to select particular parameter sets and control pump units 44 and 46 and pulse generator circuit 50 according to that parameter set. A schedule may cause pump units 44 and 46 to deliver drugs from fluid reservoirs 45 and 47 at respective times, which may include simultaneous and/or alternate delivery. For example, stimulation may be activated, deactivated, or altered at different times of the day, such as times during which the patient is awake or sleeping, or working or at rest. In addition, a schedule may electrical stimulation to be delivered in combination with drug therapy on a simultaneous or alternating basis. A clinician may create, modify, and select schedules from memory 42 using external programmer 29.

In the illustrated example of FIG. 3, electrodes 54 are electrically coupled to pulse generator 50 via electrical conductors within lead 52. In general, IMD 28 may include any number and type of electrodes. However, a greater or lesser number of electrodes may be coupled to IMD 28 to deliver electrical stimulation to patient 10. In some embodiments, a cuff electrode may provide more direct electrical contact, i.e., better electrical coupling, with a genital nerve branch or a spermatic cord than a standard ring electrode lead. However, in some cases, applying electrical stimulation directly to a nerve may result in the patient experiencing an unpleasant sensation, such as a burning sensation. Consequently, a standard ring electrode implanted proximate to the ilioinguinal nerve lead may be advantageous because the patient may experience a more pleasant paresthesia as a result of stimulation. In addition, a standard ring electrode lead may also be advantageous in terms of surgical ease.

FIGS. 7 and 9 illustrate various configurations with different types and numbers of electrodes. In general, a relatively large number of electrodes, e.g., from eight to thirty-two, may be desirable in order to permit selection of a greater number of bipolar, multipolar, and unipolar electrode combinations to deliver electrical stimulation. The availability of multiple, selectable electrode combinations increases the probability that an efficacious electrode combination can be selected.

Pulse generator 50 may comprise circuitry, such as capacitors and switches, for the generation of electrical stimulation in the form of pulses. In some embodiments, pulse generator circuit 50 may also include a switch device or switch matrix for selecting one or more electrodes for delivery of generated stimulation pulses. Accordingly, processor 40 may select one or more electrodes and the polarities of of the selected electrodes to deliver electrical stimulation to the patient. Under control of processor 40, pulse generator circuit 50 delivers the pulses to the selected electrodes via wires of lead 52 that are electrically connected to pulse generator 50. For example, as mentioned above, pulse generator 50 may include a switch device that switches stimulation pulses across selected electrodes.

IMD 28 also includes a wireless telemetry circuit 49 that allows processor 40 to communicate with external programmer 29, i.e., a clinician programmer or patient programmer. Processor 40 may receive programs to test on patient 10 from external programmer 29 via telemetry circuit 49 during programming by a clinician. Where IMD 28 stores parameter sets in memory 42, processor 40 may receive parameter sets from external programmer 29 via telemetry circuit 49 during programming by a clinician, and later receive parameter set selections made by patient 10 from external programmer 29 via telemetry circuit 49. Where external programmer 29 stores the parameter sets, processor 40 may receive parameter sets selected by patient 10 from external programmer 29 via telemetry circuit 49. In addition, processor 40 may receive parameter adjustments form external programmer 29.

The illustrated components of IMD 28 receive energy from a power source 48, such as a battery or other suitable power source. In some embodiments, power source 48 may be rechargeable and receives energy inductively captured by a recharge module (not shown). Power management circuitry (not shown) may control the recharging and discharging of power source 48. In other embodiments, power source 48 includes a nonrechargeable battery. In additional embodiments, power source 48 may receive operating power by inductive energy transfer with an external power source.

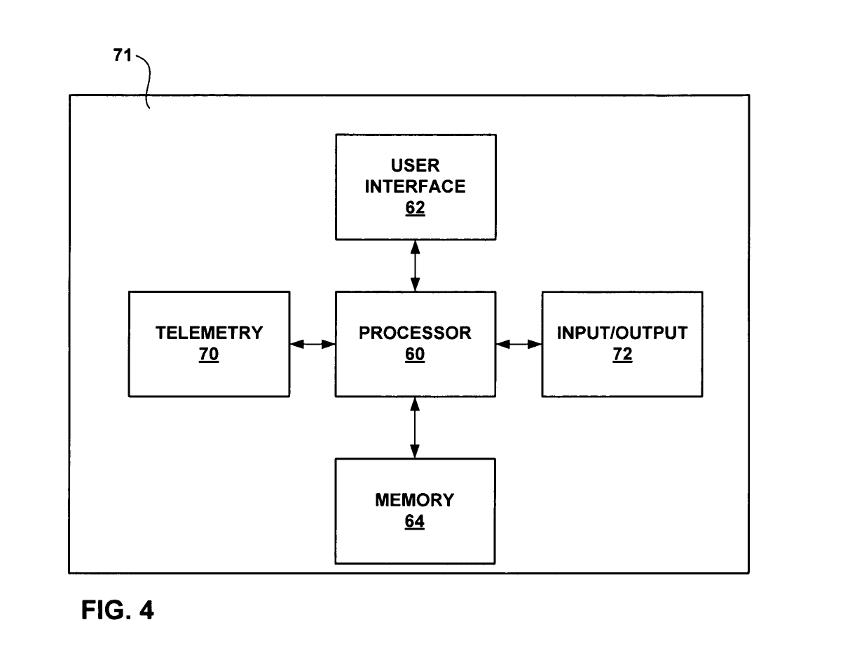

FIG. 4 is a block diagram illustrating an example patient or clinician programmer 71 that allows a patient or clinician to program drug therapy and, in some embodiments, electrical stimulation in combination with drug therapy to a genital nerve branch of a patient. Patient 10 or a clinician may interact with a processor 60 via a user interface 62 in order to control delivery of drug therapy and electrical stimulation as described herein. User interface 62 may include a display and a keypad, and may also include a touch screen or peripheral pointing devices as described above. Processor 60 may also provide a graphical user interface (GUI) to facilitate interaction with patient 10, as will be described in greater detail below. Processor 60 may include a microprocessor, a controller, a DSP, an ASIC, an FPGA, discrete logic circuitry, or the like.

Programmer 71 also includes a memory 64. In some embodiments, memory 64 may store parameter sets that are available to be selected by patient 10 or a clinician for delivery of drug therapy and electrical stimulation. Memory 64 may also store schedules. Hence, parameter sets and schedules may be stored in IMD 28, patient programmer 71, or both. Programmer 71 also includes a telemetry circuit 70 that allows processor 60 to communicate with IMD 28, and, optionally, input/output circuitry 72 that to allow processor 60 to communicate with another programmer.

Processor 60 may receive parameter set selections made by patient 10 or a clinician via user interface 62, and may either transmit the selection or the selected parameter set to IMD 28 via telemetry circuitry 70 for delivery of drug therapy and electrical stimulation according to the selected parameter set. Where programmer 71 stores parameter sets 66 in memory 64, processor 60 may receive parameter sets 66 from another programmer via input/output circuitry 72 during programming by a clinician. For example, a patient programmer may receive parameter sets from a clinician programmer. Circuitry 72 may include a transceiver for wireless communication, appropriate ports for wired communication or communication via removable electrical media, or appropriate drives for communication via removable magnetic or optical media. If wireless communication is used, telemetry circuitry 70 may support both wireless communication with IMD 28 and wireless communication with another programmer.

FIG. 5A is a schematic diagram illustrating an example system 100 for delivery of electrical stimulation in combination with one or more drugs to a male patient 10 for pelvic pain such as CTP, post vasectomy pain, genitofemoral neuralgia, and other conditions that cause long term (chronic) pain in the testicles, groin, or abdomen. System 100 also may be useful for alleviation of pelvic pain for female patients. In the illustrated example, system 100 includes one or more electrodes 104 deployed on a lead 102 extending from an IMD 108, and a fluid transfer device 106 coupled to IMD 108. Electrodes 104 and fluid transfer device 106 deliver electrical stimulation and drug therapy to spermatic cords 15 and 14, respectively, and illustrate an exemplary arrangement for delivering electrical stimulation in combination with drug therapy. However, the invention is not limited to the illustrated example. Rather, stimulation energy may be delivered to spermatic cords 14, 15 via any combination of electrodes, including axial electrode arrays, planar electrode arrays (e.g., on paddle lead), leadless microstimulators, cuff electrodes, or other types of electrodes. In addition, as previously described, electrodes 104 and fluid transfer device 106 may, in some embodiments, be implanted proximate to genitofemoral nerve 21. Following the convention illustrated in FIGS. 1 and 2, a dotted circle illustrates an example stimulation site at which electrodes 104 and fluid transfer device 106 may be implanted proximate to genitofemoral nerve 21.

IMD 108 controls the delivery of drug therapy and electrical stimulation according to preprogrammed programs, parameter sets and/or schedules. In particular, external programmer 109 may wirelessly control IMD 108 to deliver one or more drugs to spermatic cord 14 via fluid transfer mechanism 106. In the example of FIG. 5A, IMD 108 is also coupled to electrodes 104 via lead 102 that apply electrical stimulation to spermatic cord 15 under the control of IMD 108. Again, the invention is not limited to the illustrated configuration. In general, IMD 108 may be coupled to any number and type of fluid transfer devices and electrodes. The fluid transfer devices and electrodes may be positioned adjacent to one or both spermatic cords 14, 15 based on the perceived pain of patient 10. However, FIG. 5A merely illustrates example system 100 in which fluid transfer device 106 and electrodes 104 deliver bi-lateral drug therapy and electrical stimulation to spermatic cords 14 and 15.

In the illustrated example, fluid transfer device 106 is implanted adjacent to spermatic cord 14 and delivers a drug or mixture of drugs contained within IMD 108 to patient 10. As previously described, fluid transfer device 106 may include fixation elements for securing fluid transfer device 106 to tissue adjacent to spermatic cord 14 or, alternatively, directly to the external fascia of spermatic cord 14. Fixation elements may assist in keeping fluid transfer device 106 in close proximity to spermatic cord 14 as patient 10 moves. Without fixation elements, the distance between fluid transfer device 106 and spermatic cord 14 may vary through the day reducing the efficacy of the drug therapy. Fixation elements may comprise hooks, tines, barbs, helical ingrowth mechanisms, or other anchoring mechanisms. Direct contact of fluid transfer device 106 and, more particularly, fixation elements with spermatic cord 14 may be undesirable because direct contact may damage genital nerve branch 22 as patient 10 moves or if fluid transfer device 106 is removed. In addition, delivering the drug to spermatic cord 14 may result in patient 10 experiencing more complete or prolonged relief from pain and fewer unwanted side effects because the drug may affect a larger potion of tissue and nerves within spermatic cord 14.

The position of fluid transfer device 106 in FIG. 5A is for purposes of illustration. In practice, fluid transfer device 106 may be implanted above inguinal canal 27 and below genitofemoral nerve 20 to directly stimulate genital nerve branch 23 or may be implanted proximate to genitofemoral nerve 20 as indicated by the dotted circle. Delivering drug therapy at a higher position along genital nerve branch 22 (upstream in the CNS) may result in patient 10 experiencing pain relief over a larger area, which may be advantageous in some instances. In any case, fluid transfer devices are typically positioned based on the perceived pain of patient 10 and the drugs delivered to treat the pain.

IMD 108 is also coupled to electrodes 104 via lead 102 in FIG. 5A. In the example of FIG. 5A, electrodes 104 are conventional ring electrodes. In other embodiments, the electrodes may be realized by one or more cuff electrodes, as shown in FIG. 5B. In the illustrated example, electrodes 104 are connected to IMD 108 via internal electrical conductors within lead 102 and, optionally, a lead extension (not shown). The electrical stimulation delivered by electrodes 104 stimulates genital branch 22 through the fascia. The fascia protects genital nerve branch from being in direct contact with electrodes 104, thus avoiding adhesion and preventing damage to genital nerve branch 22. Electrodes 104 may also be implanted to deliver electrical stimulation directly to genital nerve branch 22. In this case, electrodes 104 may be positioned adjacent a portion of spermatic cord 15 above inguinal canal 27 and below genitofemoral nerve 23. Similar to positioning fluid transfer device 106 higher along genital nerve branch 22, positioning electrodes 104 higher along genital nerve branch 23 may result in patient 10 experiencing paresthesia over a larger area. However, genital nerve branch 23 does not include an external fascia to serve as a protective layer in this region. Consequently, placement of electrodes 104 near genital nerve branch 23 may inherently have a greater risk of pinching or otherwise damaging the nerve, possibly reducing the long-term efficacy of the electrical stimulation and the drug therapy. As a result, additional care may be necessary when positioning electrodes 104 near the genital nerve branch above the inguinal canal and below the genitofemoral nerve, as shown with respect to electrodes 104 in FIG. 5A.

System 100 generally operates in a similar manner to system 2 in FIG. 1 to deliver drug therapy to patient 10 for CTP or other pelvic pain disorders. However, unlike system 2, system 100 also delivers electrical stimulation in combination with drug therapy. Delivering electrical stimulation in combination with drug therapy may provide more complete pain relief for patient 10 or reduce and possibly prevent unwanted side effects.

External programmer 109 may be a small, battery-powered, portable device that may accompany patient 10 through the day. External programmer 109 may have a simple user interface, such as a button or keypad, and a display or lights. As shown, external programmer 109 may communicate via wireless communication with IMD 108. In particular, external programmer 109 may control delivery of drug therapy and electrical stimulation by IMD 108 using telemetry techniques known in the art. External programmer 109 may comprise a clinician programmer or a patient programmer. Where external programmer 109 comprises a patient programmer, patient 10 may only be able to active and deactivate IMD 108. Where external programmer 109 comprises a clinician programmer, external programmer 109 may include additional functionality, e.g., menus for selecting parameter sets and programs and schedules for delivering the therapy according to the selected parameters sets and programs.

FIG. 5B is a schematic diagram illustrating another exemplary arrangement of system 100 for delivering electrical stimulation in combination with drug therapy to patient 10. In particular, system 100 is illustrated in FIG. 5B as including cuff electrode 105 deployed at the distal end of lead 102 instead of electrodes 104. In the illustrated example, cuff electrode 105 applies electrical stimulation to spermatic cord 15 and fluid transfer device 106 delivers one or more drugs to spermatic cord 14 to alleviate pelvic pain in patient 10.

Cuff electrode 105 includes a cuff-like fixation structure and one or more electrodes carried by the fixation structure that deliver electrical stimulation to spermatic cord 15. Cuff electrode 105 may comprise a rigid cuff electrode, a self-sizing spiral cuff electrode, a half cuff electrode, a helical electrode, a chambered electrode, or other types of cuff electrodes that are shaped, sized and otherwise configured to at least partially wrap around spermatic cord 15. In general, cuff electrode 105 may be sized and shaped to at least partially enclose spermatic cord 15 and promote electrical coupling between the electrode and spermatic cord 15. Cuff electrode 105 may include a single or multiple electrodes. For example, cuff electrode 105 may include a bipoloar or multipolar arrangement of electrodes or a unipolar electrode that is referenced to the electrical potential of an active can electrode carried by IMD 108.

A cuff electrode may provide more direct electrical contact with a spermatic cord than a standard electrode lead. However, in some cases, applying electrical stimulation directly to a cord or nerve may result in the patient experiencing an unpleasant sensation, such as a burning sensation. Consequently, a standard electrode, such as electrodes 104 carried by lead 102, implanted proximate to the spermatic cord nerve may be advantageous because the patient may experience a more pleasant paresthesia as a result of stimulation. In addition, a standard electrode lead may also be advantageous in terms of surgical ease.

FIGS. 6A-6C are schematic diagrams illustrating an exemplary embodiment of cuff electrode 105. Cuff electrode 105 may be any type of cuff electrode used to deliver electrical stimulation, and may be deployed via a lead 102 as shown in FIG. 5B, either as an alternative to or in combination with other electrodes such as ring electrodes or paddle electrodes. In embodiments including more than one cuff electrode, the cuff electrodes may comprise the same type of cuff electrode or may comprise different types of cuff electrodes. In any case, cuff electrode 105 is merely exemplary and should not be considered limiting of the invention as broadly embodied and described in this disclosure. FIGS. 6A-6C illustrate the implantation of cuff electrodes to deliver electrical stimulation to the spermatic cord and genital branch of the genitofemoral nerve.

FIG. 6A is a top view of cuff electrode 105. Cuff electrode 105 includes lead 102, fixation structure 110, a plurality of stimulation electrodes 118A-C, and a plurality of electrical conductors 116 within lead 18. In the example of FIG. 7A, cuff electrode 105 includes three electrodes 118A, 118B, 118C. In the illustrated example, electrodes 118A-C are arranged such that a major axis of each electrode extends laterally to the spermatic cord. In this manner, the length of each electrode may be wrapped about all or a portion of the circumference of the spermatic cord. The proximal end 114 of lead 102 is connected to IMD 108 and fixation structure 110 is attached to the distal end 112 of lead 18. Cuff electrode 105may generally include one electrode or a plurality of electrodes. Each of electrodes 118A-C is coupled to one of a plurality of supply conductors 116. Electrodes 118A-C may be driven together via a common conductor or independently via separate conductors. When electrodes 118A-C are driven by a common conductor, they may be referenced to one or more electrodes carried by another lead or one or more electrodes carried by the IMD housing. When electrodes 118A-C are driven by separate conductors, bipolar or mutlipolar electrode combinations may be formed on a single lead or among two or more leads, as well as between one or more leads and the IMD housing.

For a given bipolar pair of electrodes on a lead, one supply conductor sources stimulation energy to a first electrode and a second supply conductor sinks stimulation energy from a second electrode, with the stimulation energy propagating across nerve tissue between the first and second electrodes. Hence, one electrode may form a cathode while the other forms an anode. Also, in some embodiments, multiple anodes and cathodes may be used in an electrode combination. A switch device in the IMD determines which electrodes will function as cathodes and which electrodes will function as anodes.

Fixation structure 110 may be fabricated from a flexible biocompatible material that provides a flexible interface between the electrode and the spermatic cord genital nerve branch. In some embodiments, fixation structure 110 may be fabricated from a rigid biocompatible material. The rigid fixation structure may form a split cylinder or a “U” shape sized to fit around the spermatic cord or genital nerve branch. In any case, when implanting electrode 105, the surgeon may elevate the spermatic cord and wrap fixation structure 110 around the spermatic cord or genital nerve branch. The manner in which the surgeon installs cuff electrode 105 around spermatic cord 15 or genital nerve branch depends on the type of cuff electrode. For example, if fixation structure 110 is fabricated from a shape memory alloy, fixation structure 110 may recover its shape at a fixed temperature, e.g., slightly under room temperature. By sufficiently cooling fixation structure 110, the surgeon can easily open the cuff and position fixation structure 110 under the spermatic cord. Because the nominal body temperature of the patient is above room temperature, fixation structure 110 warms up and recovers its initial shape thereby closing or wrapping fixation structure 110 around the spermatic cord. In another example, the fixation structure may be constrained in flat manner using a surgical tool or hand and, when released, wraps around the nerve.

FIG. 6B is a cross sectional view of cuff electrode 105 implanted underneath spermatic cord 15. In the illustrated example, fixation structure 110 is generally flat, thereby allowing the surgeon to easily position electrode 105under spermatic cord 15. When fixation structure 110 is fabricated from a shape memory alloy material, the surgeon may cool fixation structure 110 prior to positioning fixation structure 110 to easily manipulate fixation structure 110 into the open configuration shown in FIG. 6B. The surgeon may then position fixation structure under spermatic cord 15. Fixation structure 110 will recover its initial shape, i.e., a substantially closed ring sized to fit around spermatic cord 15, as fixation structure warms up to its activation temperature.

FIG. 6C is a cross sectional via of cuff electrode 105 implanted and wrapped around spermatic cord 15. More specifically, FIG. 7C illustrates the shape of fixation structure 110 when it has returned to its initial shape in response to warming from the patient's body heat. In the illustrated example, a gap 119 exists between spermatic cord 15 and fixation structure 110. The gap may be filled with tissue or fluids and may provide a buffer that prevents cuff electrode 105from damaging spermatic cord 15. Alternatively, fixation structure 110 may be sized to wrap around spermatic cord 15 such that there is no gap between fixation structure 110 and spermatic cord 15. In some embodiments, the fixation structure may be deployed using superelastic properties of a shape memory allow such as Nitinol. For example, the fixation structure may be constrained in a flat shape either manually or with a surgical tool, and then released so that it wraps around the nerve.

FIG. 7 is a schematic diagram further illustrating example system 100. In particular, system 100 is illustrated from the left side of a male patient 10 in FIG. 7. For purposes of illustration, only spermatic cord 15, genital nerve branch 23, femoral nerve branch 25, genitofemoral nerve 21, inguinal canal 27, and testicle 13 are shown. Again, genitofemoral nerve 21 originates from the L1 and L2 nerves in the lumbar region and divides into femoral branch 25 and genital branch 23. The dotted circle indicates an example target site for delivering drug therapy and electrical stimulation. Femoral branch 25 supplies the skin over the femoral triangle (not shown) and communicates with the intermediate cutaneous nerve (not shown) of the thigh.

In the illustrated example, fluid transfer device 106 is implanted proximate to genital nerve branch 23 and delivers a drug directly to genital nerve branch 23 and electrical stimulation is applied to spermatic cord 15 through ring electrodes 104 of lead 102 implanted adjacent to spermatic cord 15. Fluid transfer device 106 and electrodes 104 deliver drug therapy and electrical stimulation to genital nerve branch 23 under control of IMD 108.

Lead 102 carries electrodes 104 and couples electrodes 104 to IMD 108. At least one electrical conductor is included in lead 102 to electrically connect electrodes 104 to IMD 108. Typically, however, each electrode 104 will be coupled to IMD 108 via a separate conductor to permit formation of multi- and bi-polar combinations of electrodes. Electrodes 104 may comprise four electrodes, e.g., ring four electrodes, although the invention is not so limited. Electrodes 104 may comprise any number and type of four electrodes. In some embodiments, as mentioned above, lead 102 may include fixation elements, such as hooks, barbs, helical structures, tissue ingrowth mechanisms, or other anchoring mechanisms that aid in securing lead 102 to spermatic cord 15 or tissue proximate to spermatic cord 15. Securing lead 102 to spermatic cord 15 or to tissue proximate to spermatic cord 15 may prevent lead 102 from moving relative to spermatic cord 15.

IMD 108 is programmed to deliver drug therapy and electrical stimulation appropriate for CTP, post vasectomy pain, genitofemoral neuralgia, and other conditions that cause long term (chronic) pain in the testicles, groin, or abdomen. IMD 108 controls delivery of drug therapy via fluid transfer device 106 as previously described, i.e., by controlling which drug is delivered and the dosage of the drug delivered. Additionally, IMD 108 may control electrical stimulation applied by each of four electrodes 104 independently. Alternatively, IMD 108 may control electrical stimulation applied by a group of four electrodes 104, and may select different combinations of four electrodes 104 in bipolar or multi-polar arrangements to identify a particular combination that is most effective in producing desired paresthesia. Again, IMD 108 may control delivery of electrical stimulation according to parameter sets and/or schedules programmed in internal memory. Drug therapy and electrical stimulation may be applied simultaneously or on an alternating basis. In further embodiments, two leads may be deployed on opposite sides of a nerve site, so that bipolar and multipolar combinations may be formed using combinations of electrodes on both leads.

Although FIG. 7 illustrates lead 102 implanted adjacent to spermatic cord 15 below inguinal canal 27, lead 102 may be implanted similar to fluid transfer device 106, i.e., implanted adjacent to genital nerve branch 23 above inguinal canal 27. In this case, four electrodes 104 may apply electrical stimulation to genital nerve branch 23 more directly. In addition, delivering a drug to genital nerve branch 23 at a location further upstream may cause patient 10 to experience a larger area of paresthesia in response to electrical stimulation. In both male and female patients, drug therapy and electrical stimulation may be applied close or below the ilioinguinal canal 27.

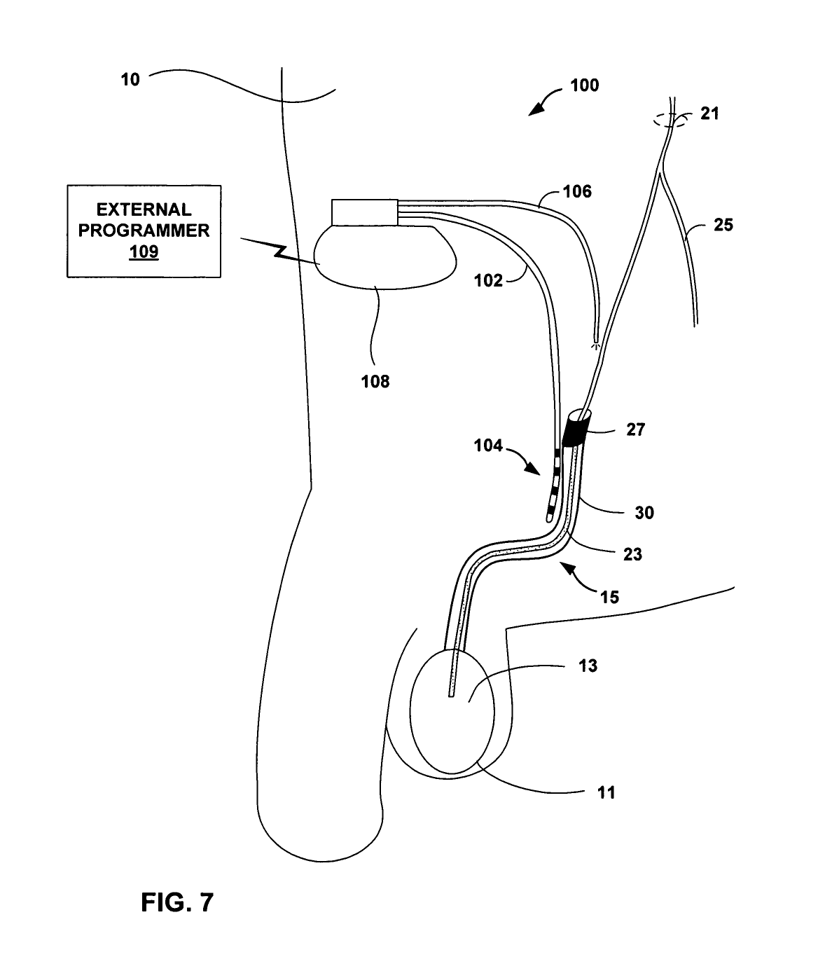

FIGS. 8A and 8B show exemplary electrical leads with fixation elements to secure the lead within a patient. As shown in FIG. 8A, lead 130 includes lead body 132, tines 136A-D (collectively tines 136) and electrodes 134A-D (collectively electrodes 134). Lead 130 may be a standard lead that includes all four tines 136 close to electrodes 134. Lead 130 may be implemented with any number of electrodes or tines. When implanting lead 130, having tines 136 close to electrodes 134 may be beneficial by allowing less movement of electrodes 134 with respect to the spermatic cord.

Electrodes 134 are more effective in delivering electrical stimulation when the electrodes are located close to the genital nerve branch spermatic cord. If electrodes 134 migrated away from the spermatic cord, due to movement of the patient throughout the day, for example, the efficacy of the stimulation may decrease. Therefore, tines 136 located close to electrodes 134 may be beneficial to therapy efficacy. An arrangement of fixation elements similar to that shown in FIG. 8A may be provided on fluid transfer devices to anchor fluid outlets adjacent to target nerve sites.

FIG. 8B illustrates a fluid delivery device 140 which includes device body 142, tines 146, and lateral fluid outlets 144A-D (collectively outlets 144). Fluid delivery device 140 alternatively, or additionally, may include a distal outlet. Fluid delivery device 140 may be a standard fluid delivery device that includes tines 146 located at the distal end of device body 142. Fluid delivery device 140 may be implemented with any number of fluid outlets or tines. Fluid outlets 144 may be located close to or a distance away from tines 146. When fluid outlets 144 are close to tines 146, implanting fluid delivery device 140 may allow less movement of fluid outlets 144 with respect to the spermatic cord.

When fluid outlets 144 are located a distance away from tines 146, implanting fluid delivery device 140 may allow fluid outlets 144 to reach further away from the anchoring site. For example, when fluid delivery device 140 delivers a drug to a genital branch of the genitofemoral nerve above the inguinal canal, i.e., before the genital nerve branch joins the spermatic cord, tines may be anchored to tissue a distance away from the genital nerve branch while outlets 144 may be located proximate to the genital nerve branch. Securing tines 146 to genital nerve branch is undesirable because the nerve may be damaged in the process. Thus, fluid delivery device 140 may be beneficial by preventing unwanted nerve damage during the implantation process. An arrangement of fixation elements similar to that shown in FIG. 8B may be provided on electrical stimulation leads to anchor electrodes adjacent to target nerve sites.

FIG. 9 is a schematic diagram further illustrating example system 100. In the example of FIG. 9, system 100 includes a leadless microstimulator 150, e.g., as an alternative to a ring electrode lead. System 100 is illustrated from the right side of a male patient 10 in FIG. 9. For purposes of illustration, only spermatic cord 14, genital nerve branch 22, femoral nerve branch 24, genitofemoral nerve 20, inguinal canal 26, and testicle 12 are shown. As previously described, spermatic cord 14 includes various layers and structures. For example, as spermatic cord 14 passes through inguinal canal 26, it joins the cremasteric layer of muscle and fascia responsible for the cremasteric reflex. Additionally, spermatic cord 14 picks up an external fascia layer 32 as it exits inguinal canal 26 through the superficial ring. Accordingly, genital branch 22 is shown within the external fascia 32 of spermatic cord 14.

In the illustrated example, fluid transfer device 106 is implanted proximate to genital nerve branch 22 and delivers a drug directly to genital nerve branch 22 and microstimulator 150 applies electrical stimulation to spermatic cord 14. Fluid transfer device 106 and microstimulator 150 delivery drug therapy and electrical stimulation, respectively, to genital nerve branch 22 under control of IMD 108. In some embodiments, microstimulator 150 may be controlled by IMD 108 or external programmer 109 via wireless telemetry. In other embodiments, microstimulator 150 may operate autonomously, subject to reprogramming or parameter adjustment by external programmer 109.

As shown, IMD 108 or external programmer 109 may wirelessly control microstimulator 106 to deliver electrical stimulation. In the example of FIG. 9, microstimulator 150 includes a housing 154 and a fixation structure 152, such as a cuff, attached to housing 154. Housing 154 may be formed into a capsule-like shape and may be constructed from any of a variety of biocompatible materials, such as titanium or stainless steel. Housing 154 may carry an implantable pulse generator (IPG) and a telemetry interface to exchange (send, receive, or both) control signals with IMD 108, external programmer 109, or both. Fixation structure 152 wraps at least partially around spermatic cord 14 to secure microstimulator 150 in place. Accordingly, fixation structure 152 may operate and be constructed of a flexible or rigid biocompatible material similar to the fixation structure of previously described cuff electrode 104. Fixation structure 152 may carry one or more electrodes, i.e., the electrodes may be integrated with fixation structure 152, and housing 154 may include short leads (not shown) that extend from housing 154 to couple the electrodes to housing 154. In some embodiments, housing 154 may form an active “can” electrode.

Microstimulator 150 may be implanted with less invasive procedures than electrodes that are coupled to an IMD via a lead. For example, because microstimulator 150 wirelessly communicates with IMD 108, a surgeon does not have to tunnel a lead to IMD 108. In some embodiments, microstimulator 150 may wirelessly communicate with external programmer 109.

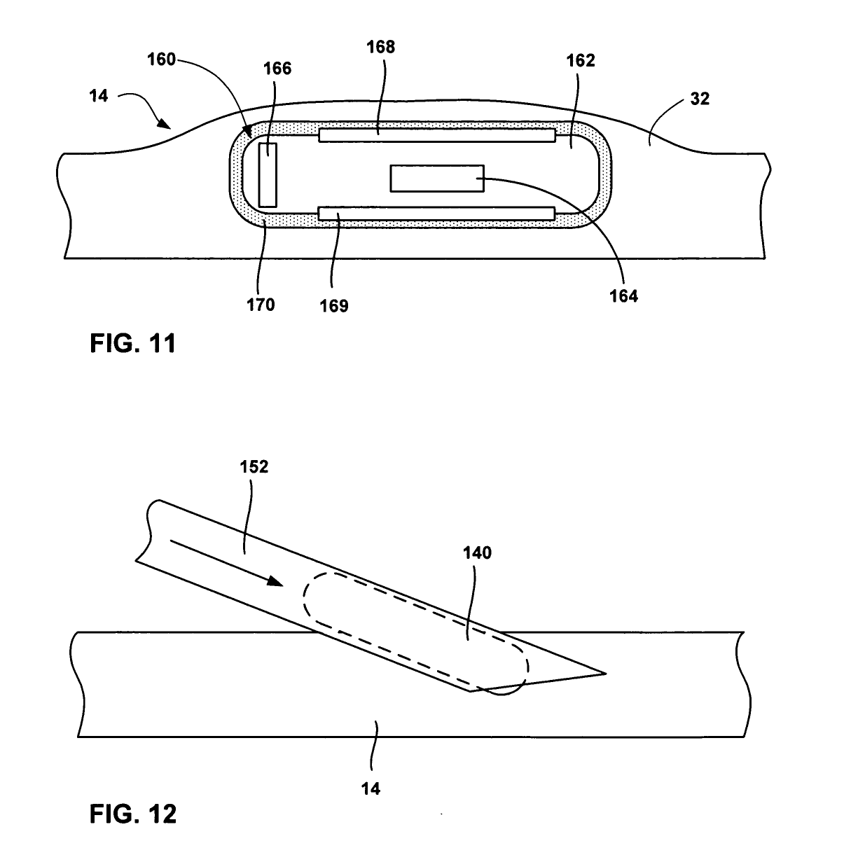

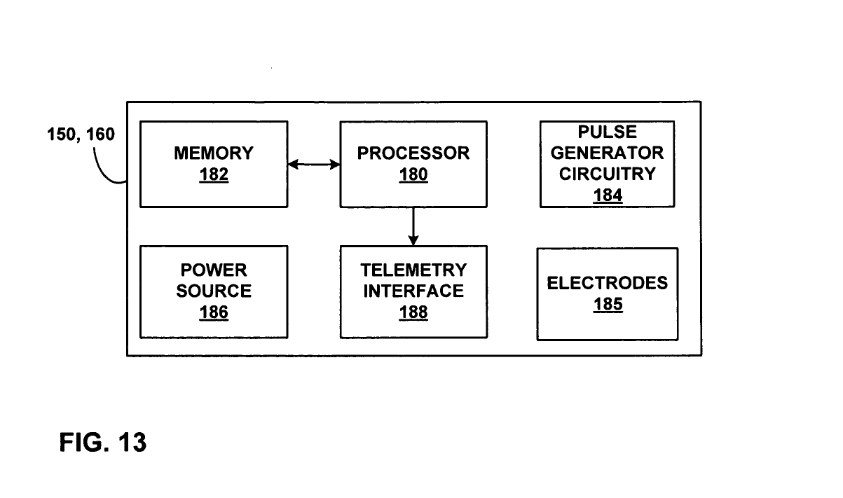

Microstimulator 150 may also be implanted within the external fascia of spermatic cord 14 using a needle (not shown) as illustrated in FIGS. 12 and 13. In this case, microstimulator 150 may be implanted with a minimally invasive, percutaneous procedure. As an example, the needle may include a hollow cylinder and a pointed distal end for puncturing skin of patient 10. The needle may include the microstimulator and a fluid, e.g., saline solution, or push rod to force the microstimulator out of the needle. In this case, microstimulator 150 may be miniaturized in order to be implanted using the needle. In some embodiments, a plurality of microstimulators may be implanted within the external fascia of the spermatic cord or in tissue proximate to the genital branch of the genitofemoral nerve. The plurality of implanted microstimulators may apply electrical stimulation independently or on a coordinated basis.

When implanted within the external fascia of the spermatic cord, microstimulator 150 may comprise a self-contained module. The module comprises a housing that may carry one or more electrodes and an IPG within the housing. The IPG may comprise a circuit board and a power source, such as a battery, to provide power to the circuit board and electrodes. The circuit board may include the telemetry interface and other processing electronics. The electrodes may be pads mounted on a surface of the housing or ring electrodes that extend about the entire periphery of the housing. In some cases, the housing itself may form an active “can” electrode in addition to the electrodes mounted on the housing.

The invention is not limited to the illustrated configuration. In general, fluid transfer device 106 and microstimulator 150 may be implanted in any combination at various sites along genital nerve branch 22 or genitofemoral nerve 20. Furthermore, any number of fluid transfer devices and microstimulators or other types of electrodes may be implanted in any combination to provide uni-lateral or bi-lateral pain relief. As an example, microstimulator 150 may be implanted similar to fluid transfer device 106 to deliver electrical stimulation in combination with drug therapy to genital nerve branch 22 above inguinal canal 26. In this case, fixation structure 154 may wrap at least partially around genital nerve branch 22. When delivering a drug to genital nerve branch 22 before it joins spermatic cord 14, microstimulator 150 may be beneficial because it does not require fixation elements to secure it in place and, therefore, may not damage genital nerve branch 22. In addition, in some embodiments, a microstimulator may be implanted to deliver electrical stimulation at both locations in a coordinated manner or independently of each other. In further embodiments, a microstimulator may also be implanted in proximate to genitofemoral nerve 20. A microstimulator may be implanted proximate to genitofemoral nerve 20 using techniques similar to implanting microstimulator proximate to genital nerve branch 22. The dotted circle around genitofemoral nerve 20 indicates an example site at which microstimulator 150 may be implanted.

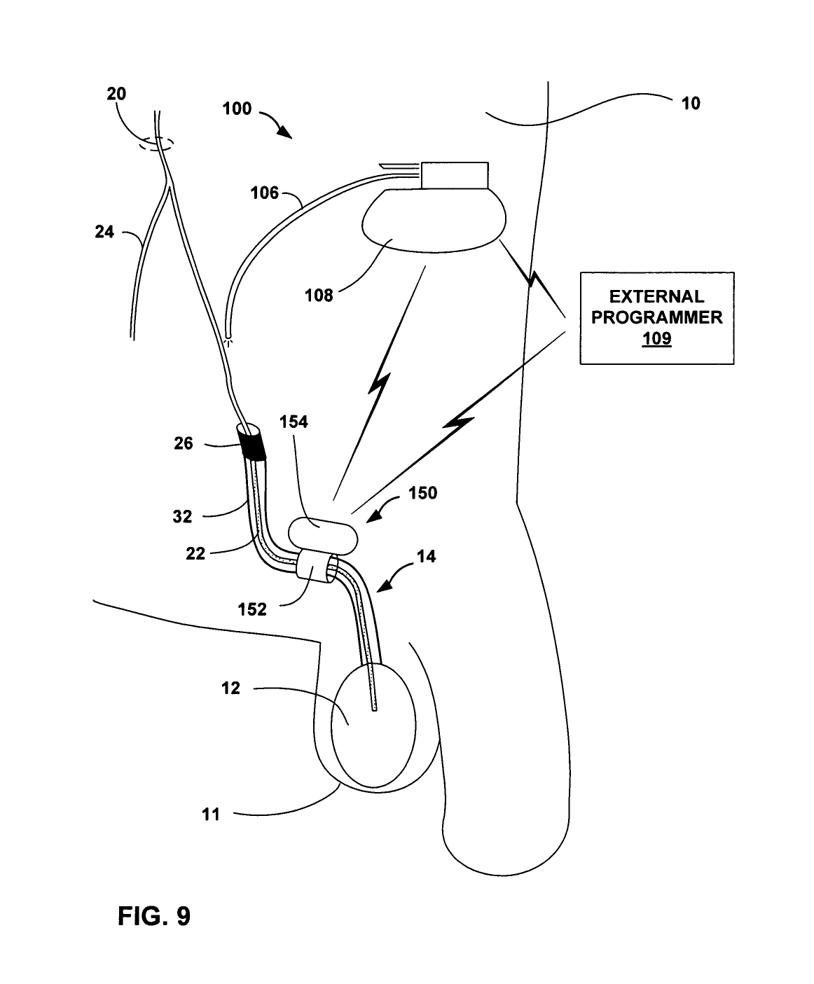

FIGS. 10A-10C are enlarged schematic diagrams showing microstimulator 150. In particular, FIG. 10A is an enlarged top view of microstimulator 150 including housing 154, circuit board 156, power supply 155, fixation structure 152, and electrodes 158A-C (collectively electrodes 158). Housing 154 may have a rounded, capsule-like shape, and a smooth, atraumatic surface formed of one or more biocompatible materials, such as titanium, stainless steel, epoxy, or polyvinylchloride. However, the invention is not so limited. Instead, housing 154 may have a shape that is compatible with the anatomy at the implant site, i.e., the spermatic cord or the genital nerve branch before it joins the spermatic cord. In some embodiments, the leadless microstimulator may have a capsule shape with a diameter of less than or equal to approximately 2 cm and a length of less than or equal to approximately 5 cm.

Fixation structure 152 may be constructed of a flexible or rigid biocompatible material that at least partially wraps around the spermatic cord or genital nerve branch, e.g., like a cuff. For example, fixation structure 152 may be fabricated from a shape memory alloy that has the capacity to recover a memorized shape when deformed at a certain temperature and then heated at a higher temperature or vice versa. In this case, the memorized shape may be a split cylinder or a substantially closed cylinder with a diameter sized to wrap around the spermatic nerve or genital nerve branch.

FIG. 10A illustrates fixation structure 152 in a deformed, generally open state that enables a surgeon to easily position slip microstimulator 150 underneath the spermatic cord. However, after positioning microstimulator 150 beneath the spermatic cord, the body temperature of the patient causes fixation structure 152 to recover its memorized shape, i.e., a split cylinder. Therefore, fixation structure 152 may be beneficial by reducing trauma during surgical implantation procedures.

Fixation structure 152 also carries one or more electrodes 158. Electrodes 158 may be driven together or independently. Electrodes 158 may be integrated with fixation structure 152 or, alternatively housing 154 may include short leads (not shown) that extend from housing 154 to couple electrodes 158 to housing 154.

Circuit board 156 may include a processor, memory, pulse generator circuitry to generate electrical pulses delivered by IMD 108, and telemetry circuitry for wireless telemetry with IMD 108, external programmer 109, or both. As an example, the memory may store stimulation parameters, e.g., electrode polarity, pulse width, pulse rate, and amplitude. Memory may also store schedules which define times for the processor to select particular parameters. A schedule may cause electrical stimulation to be delivered at respective times. In this manner, the processor may control the pulse generator circuitry to generate electrical stimulation pulses in accordance with the selected parameters and schedule.

Microstimulator 150 may also operate under control from an external programmer, so that a physician or patient may activate, deactivate and/or modify stimulation delivered to the patient on a selective basis. Power source 155 supplies operating power to circuit board 156 and may take the form of a small rechargeable or non-rechargeable battery. Different types of batteries or different battery sizes may be used. To promote longevity, power source 155 may be rechargeable via induction or other means.

FIG. 10B illustrates a cross sectional view of microstimulator 150 implanted underneath spermatic cord 14. In the illustrated example, fixation structure 152 is flat, thereby allowing the surgeon to easily position microstimulator 150 underneath spermatic cord 14. When fabricated from an shape memory alloy, the body temperature of patient 10 may heat fixation structure 152 above the recovery shape temperature.

FIG. 10C is a cross sectional view of microstimulator 150 with fixation structure 152 wrapped substantially around spermatic cord 14. For example, as fixation structure 152 is warmed above its recovery shape temperature, fixation structure 152 recovers its initial shape, i.e., a substantially closed cylinder or ring. As shown in FIG. 10C, in some embodiments, fixation structure 152 may not close completely. However, fixation structure 152 may at least wrap partially around spermatic cord 14, or the genitofemoral nerve or genital nerve branch in order to secure microstimulator 150 to the nerve site. Removing microstimulator 150 may be easier when fixation structure 152 does not completely wrap around spermatic cord 14 because the gap between the ends of fixation structure 152 may provide an area to insert a tool that aids in removal. In alternative embodiments, fixation structure 152 may wrap completely around spermatic cord 14.

In the illustrated example, a gap 109 exists between spermatic cord 14 and fixation structure 152. Gap 109 may be filled with tissue or fluids and may provide a buffer that prevents microstimulator 150 from damaging spermatic cord 14. Alternatively, fixation structure 152 may be sized to wrap around spermatic cord 14 such that there is no gap between fixation structure 152 and spermatic cord 14.

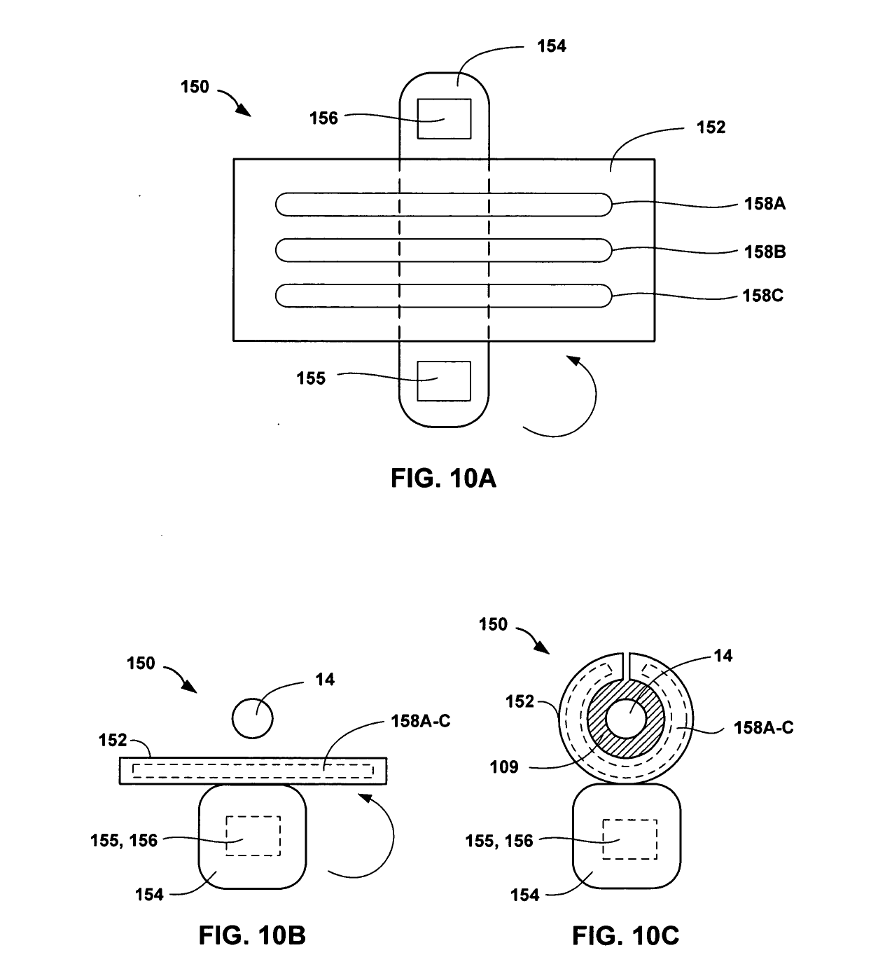

FIG. 11 is cross-sectional view of a microstimulator 160 implanted within, for example, spermatic cord 14. Housing 162 of microstimulator 160 is embedded in the external fascia 32 of spermatic cord 14 and includes circuit board 164, power source 166, and electrodes 168 and 169. Housing 162 is in the shape of a rounded capsule and includes a smooth surface. The only structure extending from housing 162 are electrodes 168 and 169. Electrodes 168 and 169 may protrude slightly from housing 162 or, alternatively, may be integrated into housing 162 to apply electrical stimulation to external fascia 32. Microstimulator 160 rests in wall cavity 170 formed within external fascia 32. As previously described, microstimulator 160 may have a cylindrical shape with a diameter of less than or equal to approximately 2 cm and a length of less than or equal to approximately 5 cm.

Circuit board 164, power source 166, and electrodes 168 and 169 may be similar to respective circuit board 156, power source 155, and electrodes 108 of FIGS. 11A-C. Differences between these components of each embodiment may relate to the size or shape of each component. Therefore, electrodes 168 and 169 apply electrical stimulation under control of circuit board 164. Power source supplies operating power to circuit board 164. Circuit board 164 may select may select stimulation parameters and cause electrodes 168 and 169 to apply electrical pulses with the selected parameters according to schedules stored in memory. Circuit board 160 receives control signals from IMD 108, external programmer 109, or both by wireless telemetry. In some embodiments, one of electrodes 168 and 169 may comprise a sensor or microstimulator 160 may additionally include a sensor that detects a physiological parameter. In such embodiments, the sensor may sense a change in a physiological parameter. Processing electronics on circuit board 164 detects the change and causes electrodes 168 and 69 to apply electrical stimulation in response to the change.

Implanting microstimulator 160 within external fascia 32 of spermatic cord 14 may be a simple method for securing electrodes 168 and 169. Microstimulator 160 may also be implanted in tissue proximate to genital nerve branch 22 or implanted in tissue proximate to genitofemoral nerve 20. In some embodiments, a plurality of microstimulators similar to microstimulator 160 may be implanted and apply electrical stimulation to spermatic cord 14 in a coordinated manner or in a manner independent of each other.

FIG. 12 is a schematic diagram illustrating implantation of microstimulator 160 within the spermatic cord 14. Microstimulator 160 may be implanted through endoscopic, laparoscopic, or similar minimally invasive techniques. A surgeon may make a small inguinal incision in patient 10 and guides microstimulator 160 within needle 172 to spermatic cord 14. Needle 172 may be constructed of a metal alloy and comprise a hollow cylinder and a pointed distal end for puncturing the skin of patient 10. Needle 172 includes microstimulator 160 and a fluid or push rod to force microstimulator 160 out of the needle. An exemplary fluid may be saline or other biocompatible fluid.

Once needle 172 in positioned at the appropriate location with respect to spermatic cord 14, the surgeon may force microstimulator 160 into place. Removing needle 172 from spermatic cord 14 allows the external fascia of spermatic cord 14 to close and surround microstimulator 160. When implanting microstimulator 160, the external fascia should not be breached in order to prevent other structures within spermatic cord 14, such as the genital nerve branch, ductus deferens, lymph vessels, pampiniform plexus of veins which become the testicular vein, and testicular artery, from being damaged.

In other embodiments, microstimulator 160 may be implanted through more invasive procedures which expose spermatic cord 14. As previously described, multiple microstimulators may be implanted with spermatic cord 14 to apply electrical stimulation to a larger area. Microstimulator 160 may also be implanted within tissue proximate to the genital branch of the genitofemoral nerve.

FIG. 13 is a functional block diagram illustrating various components of an example microstimulator 150 (FIG. 9) or microstimulator 160 (FIG. 11). In the example of FIG. 13, microstimulator 150, 160 includes a processor 180, memory 182, pulse generator circuitry 184, telemetry interface 188, power source 186 and electrodes 185. Pulse generator circuitry 184 may be carried on a circuit board, along with processor 180, memory 182, and telemetry interface 188. Memory 182 may store instructions for execution by processor 180, stimulation parameters, e.g., electrode polarity, pulse width, pulse rate, and amplitude, and schedules for delivering electrical stimulation. Memory 182 may include separate memories for storing instructions, stimulation parameter sets, and schedules. Memory 182 may comprise any form of computer-readable media such as magnetic or optical tape or disks, solid state volatile or non-volatile memory, including random access memory (RAM), read only memory (ROM), electronically programmable memory (EPROM or EEPROM), or flash memory.

Processor 180 controls pulse generator circuitry 184 to deliver electrical stimulation via electrodes 185. Electrodes 185 may comprise any number and type of electrodes previously described, i.e., electrodes 158 (FIG. 9) and electrodes 168 and 169 (FIG. 11). An exemplary range of stimulation pulse parameters likely to be effective in treating, post vasectomy pain, genitofemoral neuralgia, and other conditions that cause long term pain in the testicles, groin, or abdomen when applied to the spermatic cord or genital nerve branch are as follows: pulse widths between approximately 10 and 5000 microseconds, more preferably between approximately 100 and 1000 microseconds and still more preferably between 180 and 450 microseconds; voltage amplitudes between approximately 0.1 and 50 volts, more preferably between approximately 0.5 and 20 volts and still more preferably between approximately 1 and 10 volts; and frequencies between approximately 0.5 and 500 hertz, more preferably between approximately 10 and 250 hertz and still more preferably between approximately 50 and 150 hertz. The pulses may be alternating current (ac) pulses or direct current (dc) pulses, and may be mono-phasic, bi-phasic, or multi-phasic in various embodiments. The above parameters may be applicable to stimulation delivered by microstimulators, paddle lead electrode arrays, ring electrode leads, or other stimulation electrodes.

Processor 180 also controls telemetry interface 188 to receive information from IMD 108, external programmer 109, or both. Telemetry interface 188 may communicate via wireless telemetry, e.g., RF communication, on a continuous basis, at periodic intervals, or upon request from the implantable stimulator or programmer. Processor 180 may include a single or multiple processors that are realized by microprocessors, Application-Specific Integrated Circuits (ASIC), Field-Programmable Gate Arrays (FPGA), or other equivalent integrated or discrete logic circuitry.

Power source 186 delivers operating power to the components of the implantable microstimulator. As mentioned previously, power source 186 may include a small rechargeable or non-rechargeable battery and a power generation circuit to produce the operating power.

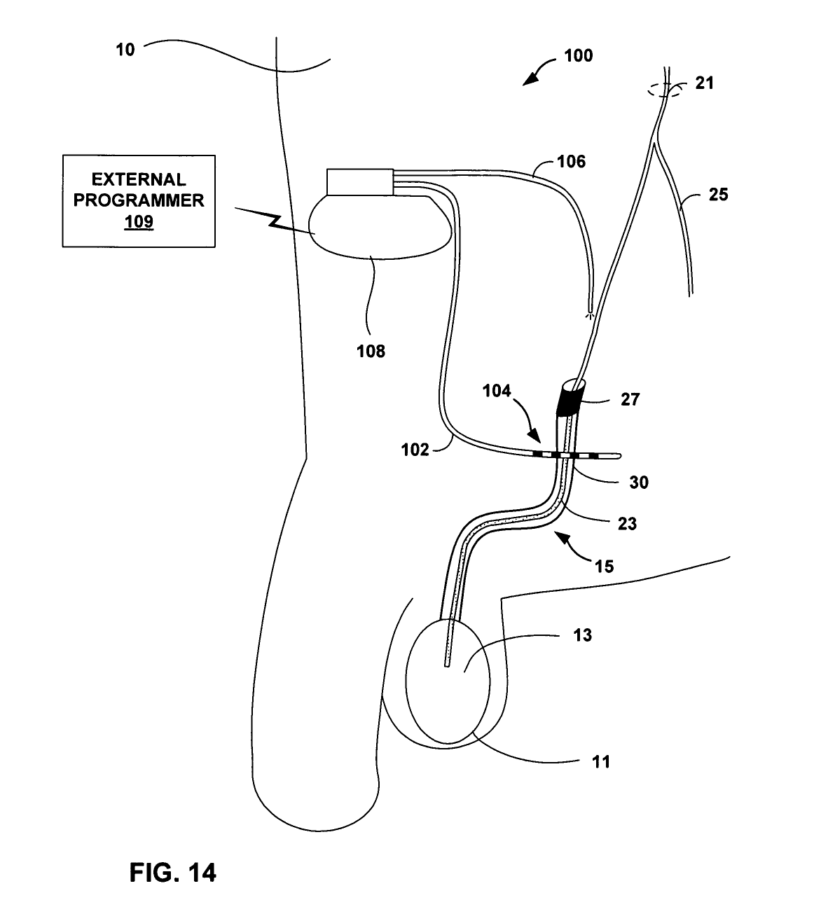

FIG. 14 is a schematic diagram illustrating another configuration for example system 100. In particular, rather than being implanted along spermatic cord 15, electrode 124 is illustrated in FIG. 14 as being implanted perpendicular to spermatic cord 15. Implanting electrode 124 perpendicular to spermatic cord 15 may provide certain advantages. For example, when implanted as shown, electrode 124 may more effectively apply electrical stimulation to a point along spermatic cord 15 instead of applying electrical stimulation along a length or portion of spermatic cord 15. Patient 10 may experience a more complete relief of pain or fewer unwanted side effects as a result of applying electrical stimulation in this manner. The invention is not limited to the illustrated embodiments. Instead, electrode 124 may be implanted at any orientation with respect to spermatic cord 15.

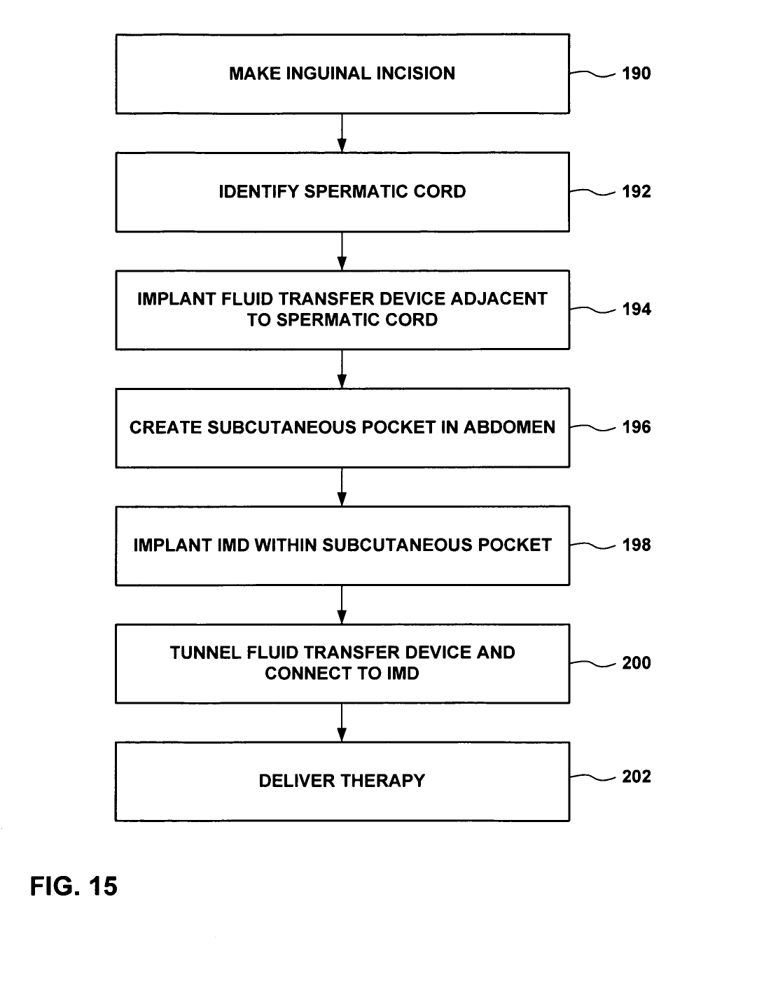

FIG. 15 is a flow chart illustrating a technique for delivering a drug to a spermatic cord of a patient using an IMD including an drug delivery device. The IMD may include any number of fluid transfer devices and, in some embodiments, may also include an electrical stimulation device. In such embodiments, any of the previously described electrodes, i.e., a cuff electrode 105 (FIGS. 5B and 6A-6C), electrodes 104 carried by lead 102 (FIGS. 5A, 7, and 14), microstimulator 150 (FIG. 9), and microstimulator 160 (FIG. 11), may be implanted and deliver electrical stimulation in combination with drug therapy in accordance with the steps of the illustrated flow chart. The flow of events begins with the surgical procedure for implanting the fluid transfer devices. The surgical procedure for exposing the spermatic cord is well defined and may be used. Specifically, the surgeon makes an inguinal incision (190) as used for standard spermatic denervation or hernia repair.

The surgeon identifies the spermatic cord (192) and implants a fluid transfer device adjacent to the spermatic cord (194). Where the fluid transfer device includes fixation elements, such as tines, barbs, and other anchoring mechanisms, the surgeon may secure the fixation elements to tissue adjacent to the spermatic cord to avoid damage to the spermatic cord and prevent the fluid transfer device from shifting as the patient moves. If the fluid transfer device includes a fixation element similar to the cuff of cuff electrode 105 (FIGS. 6A-6C), the surgeon may elevate the spermatic cord and wrap the cuff around the spermatic cord. If the fixation structure of the spermatic cord is formed from a shape memory alloy, the body temperature of the patient may cause the fixation structure to recover its initial shape, i.e., a substantially closed cylinder or ring shape sized to fit around the spermatic cord. In any case, the cuff may wrap at least partially around the spermatic cord thereby securing the fluid transfer device to the spermatic cord.

In embodiments in which electrical stimulation is applied to a genital nerve branch in combination with drug therapy, the surgeon may implant electrodes using a method similar to implanting fluid transfer devices. For example, when implanting a lead carrying electrodes, fixation elements may secure the lead to the spermatic cord or tissue proximate to the spermatic cord. Leads carrying electrodes may provide distinct advantages over leadless stimulators due to the number of electrodes available to apply electrical stimulation. For example, leads are available that carry eight, sixteen, or more electrodes which can be used to applying electrical stimulation in various groups or independently of each other. Further, because the electrodes may be positioned along a substantial length of the lead, the electrodes may apply electrical stimulation along a larger area of the spermatic cord.

Using a microstimulator, e.g., microstimulator 150 (FIG. 9), as an example of a leadless stimulator, the surgeon may implant microstimulator 150 similar to cuff electrodes, e.g., cuff electrode 105 (FIGS. 6A-6C), or a fluid transfer device with a cuff fixation structure because the fixation structure of microstimulator 106 may operate in the same manner as the fixation structure of cuff electrode 105. In contrast, the surgeon may implant microstimulator 160 (FIG. 11) within the external fascia of the spermatic cord using a needle. The needle may comprise a hollow cylinder and a pointed distal end for puncturing the skin of the patient and a fluid to force microstimulator 150 out of the needle. Accordingly, the surgeon may not need to make an inguinal incision when implanting microstimulator 150 within the external fascia of the spermatic cord. Rather, once the needle is positioned at the appropriate location with respect to the spermatic cord, the surgeon forces microstimulator 150 into place by depressing the plunger of the needle thereby forcing the fluid and microstimulator out of the needle.

Removing the needle from the spermatic cord allows the external fascia of the spermatic cord to close and surround microstimulator 150. Consequently, microstimulator 150may be implanted with a minimally invasive surgical procedure. Additionally, in some embodiments, the surgeon may implant a plurality of microstimulators along the spermatic cord. The microstimulators may provide electrical stimulation independently or on a coordinated basis.

Although the implantation techniques have been described with respect to the spermatic cord, the implantation techniques may also be used to implant fluid transfer devices and electrodes adjacent to the genital branch of the genitofemoral nerve. In particular, the surgeon may implant the fluid transfer device adjacent to the genital nerve branch before it joins the spermatic cord. Implanting a fluid transfer device adjacent to the genital nerve branch in this manner may provide paresthesia to a larger area of the patient because electrical stimulation is applied upstream of the spermatic cord.

In any case, after implanting the fluid transfer device, the surgeon may create a subcutaneous pocket in the abdomen or buttock of the patient (196) and implant an IMD, such as IMD 28 (FIG. 1) or IMD 108 (FIGS. 5A and 5B), within the subcutaneous pocket (198). In some embodiments, the IMD may be miniaturized and implanted within the scrotum of the patient. The surgeon may then tunnel the fluid transfer device lead through the patient to the implantation site and connect the fluid transfer device to the IMD (200). Notably, in embodiments that deliver electrical stimulation in combination with drug therapy, microstimulators 150 and 160 may wirelessly communicate with external programmer 109 to receive control signals and, thus, do not require an IMD.

When the surgical implantation procedure is complete, the implanted fluid transfer devices may deliver drug therapy (202), i.e., one or more drugs, to the spermatic cord or, alternatively, the genital nerve branch. Delivering a drug to the spermatic cord may block pain signals from the testicles and the associated scrotal area from reach the central nervous system. The pain experienced by the patient may be uni-lateral or bi-lateral. Consequently, fluid transfer devices may be implanted adjacent to one or both spermatic cords of a patient. The pain experienced by the patient may also be constant or intermittent, or spontaneous or exacerbated by physical activities and pressure. Thus, the implanted fluid transfer devices may deliver drugs on demand, such as in response to a control signal received from a patient or clinician programmer, or in accordance with preprogrammed cycles or schedules.

Delivering drug therapy to the genitofemoral nerve or the genital nerve branch may provide may provide substantial relief of pelvic pain experienced by male and female patients, including urogenital pain or other forms of pelvic pain. In male patients, for example, delivering drug therapy to the genitofemoral nerve or the genital nerve branch (directly or via the spermatic cord) may relieve a variety of pelvic pain conditions such as chronic testicular pain (CTP), post vasectomy pain, genitofemoral neuralgia, and other conditions that cause long term (chronic) pain in the testicles, groin, or abdomen. For female patients, delivering drug therapy to the genitofemoral nerve or the genital nerve branch may alleviate a variety of pelvic pain conditions such as pain resulting from surgical procedures, vulvodynia, interstitial cystitis (painful bladder syndrome), adhesions, endometriosis, and pelvic congestion. Accordingly, although the invention has been primarily described with respect to male patients, the invention is not so limited and may be readily applied to female patients for similar relief of pain symptoms.

The invention is not limited to delivering only drug therapy. Rather, the invention also describes embodiments that deliver electrical stimulation in combination with drug therapy to one or both genital nerve branches, directly or indirectly via the spermatic cord. Electrical stimulation and drug therapy may be delivered simultaneously or on an alternating basis. For example, drug therapy may be delivered constantly or intermittently through the course of a day and the patient may use a patient programmer to deliver electrical stimulation when experiencing moments of increased pain. Alternatively, electrical stimulation may be delivered according to preprogrammed parameter sets and schedules and the patient may use a patient programmer to deliver drug therapy when the electrical stimulation does not substantially reduce the pain.

The techniques described in this disclosure may be implemented in hardware, software, firmware or any combination thereof. For example, various aspects of the techniques may be implemented within one or more microprocessors, digital signal processors (DSPs), application specific integrated circuits (ASICs), field programmable logic arrays (FPGAs), or any other equivalent integrated or discrete logic circuitry, as well as any combinations of such components. The term “processor” or “processing circuitry” may generally refer to any of the foregoing logic circuitry, alone or in combination with other logic circuitry, or any other equivalent circuitry.

When implemented in software, the functionality ascribed to the systems and devices described in this disclosure may be embodied as instructions on a computer-readable medium such as random access memory (RAM), read-only memory (ROM), non-volatile random access memory (NVRAM), electrically erasable programmable read-only memory (EEPROM), FLASH memory, magnetic media, optical media, or the like. The instructions are executed to support one or more aspects of the functionality described in this disclosure

Many embodiments of the invention have been described. Various modifications may be made without departing from the scope of the claims. For example, although delivery of one or more drugs has been described, other fluids may be delivered in addition, or as an alternative, to such drugs. Such fluids may include, for example, saline, biological fluids, gene therapy suspensions or cultures, or the like. These and other embodiments are within the scope of the following claims.

1. A method comprising delivering a drug to a genital nerve branch of a genitofemoral nerve of a patient via an implanted drug delivery device.

2. The method of claim 1, wherein the patient is a male patient, the method further comprising delivering the drug to the genital nerve branch via a spermatic cord of the patient.

3. The method of claim 1, wherein the patient is a male patient, the method further comprising delivering the drug to the genital nerve branch at a point on a side of an inguinal canal of the patient opposite a spermatic cord of the patient.

4. The method of claim 1, wherein the patient is a male patient, the method further comprising delivering the drug to the genital nerve branch at a point on a side of an inguinal canal of the patient adjacent a spermatic cord of the patient.

5. The method of claim 1, further comprising delivering the drug to first and second genital nerve branches of first and second genitofemoral nerves of a patient via the implanted drug delivery device.

6. The method of claim 5, wherein the patient is a male patient, the method further comprising delivering the drug to the first and second genital nerve branches via first and second spermatic cords of the patient.

7. The method of claim 5, wherein the patient is a male patient, the method further comprising delivering the drug to the first and second genital nerve branches at points on a side of respective inguinal canals of the patient opposite respective spermatic cords of the patient.

8. The method of claim 5, wherein the patient is a male patient, the method further comprising delivering the drug to the first and second genital nerve branches at points on a side of respective inguinal canals of the patient adjacent respective spermatic cords of the patient.

9. The method of claim 1, wherein the drug is selected to alleviate pelvic pain.

10. The method of claim 9, wherein the pelvic pain includes at least one of chronic testicular pain, post vasectomy pain, genitofemoral neuralgia, vulvodynia, and interstitial cystitis.

11. The method of claim 1, wherein the drug comprises at least one of gabapentin, morphine, clonidine, tizanidine, hydromorphone, fentanyl, sufentanil, methadone, meperidine, tetracaine, bupivicaine, zinconotide, adenosine, ketorolac, baclofen, ropivicaine, ketamine, octreotide, neostigmine, and droperidol.

12. The method of claim 1, wherein the implanted drug delivery device comprises a reservoir for storing the drug and a fluid transfer device coupled to the reservoir, and wherein delivering the drug comprises delivering the drug from the reservoir to the genital nerve branch via the fluid transfer device.

13. The method of claim 1, further comprising delivering electrical stimulation to the genital nerve branch of the patient via an implanted electrical stimulation device.

14. The method of claim 13, wherein delivering electrical stimulation comprises delivering electrical stimulation to first and second genital nerve branches of first and second genitofemoral nerves of the patient via the implanted electrical stimulation device.

15. The method of claim 13, wherein the electrical stimulation is selected to alleviate pelvic pain.

16. A system comprising:
an implantable drug delivery device that delivers a drug selected to alleviate pelvic pain to a genital nerve branch of at least one genitofemoral nerve of a patient; and
an implantable electrical stimulation device that delivers electrical stimulation selected to alleviate pelvic pain to a genital nerve branch of at least one genitofemoral nerve of the patient.

17. The system of claim 16, wherein the drug is selected to alleviate pelvic pain including at least one of chronic testicular pain, post vasectomy pain, genitofemoral neuralgia, vulvodynia, and interstitial cystitis.

18. The system of claim 16, wherein the drug comprises at least one of gabapentin, morphine, clonidine, tizanidine, hydromorphone, fentanyl, sufentanil, methadone, meperidine, tetracaine, bupivicaine, zinconotide, adenosine, ketorolac, baclofen, ropivicaine, ketamine, octreotide, neostigmine, and droperidol.

19. The system of claim 16, wherein the implantable drug delivery device comprises:
a reservoir that stores the drug;
a fluid transfer device to transfer the drug from the reservoir to the genital nerve branch, the fluid transfer device having a proximal end for receiving the drug from the reservoir and a distal end for delivering the drug to the delivery site; and
a pump unit coupling the reservoir to the proximal end of the fluid transfer device that causes the transfer of the drug from the reservoir to the delivery site via the fluid transfer device.

20. The system of claim 16, further comprising a processor that controls both the drug delivery device and the electrical stimulation device.

21. The system of claim 16, wherein the drug delivery device and the electrical stimulation device include a common housing.

22. The system of claim 16, wherein the patient is a male patient, and wherein the fluid transfer device is positioned to deliver the drug to the genital nerve branch via a spermatic cord of the patient.

23. The system of claim 16, wherein the patient is a male patient, and wherein the fluid transfer device is positioned to deliver the drug to the genital nerve branch at a point on a side of an inguinal canal of the patient opposite a spermatic cord of the patient.

24. The system of claim 16, wherein the patient is a male patient, and wherein the fluid transfer device is positioned to deliver the drug to the genital nerve branch at a point on a side of an inguinal canal of the patient adjacent a spermatic cord of the patient.

25. A method comprising delivering a drug to at least a portion of a genitofemoral nerve of a patient via an implanted drug delivery device.

26. The method of claim 25, further comprising delivering the drug to first and second genitofemoral nerves of the patient via the implanted drug delivery device.

27. The method of claim 25, wherein the drug is selected to alleviate pelvic pain.

28. A system comprising:
an implantable drug delivery device that delivers a drug selected to alleviate pelvic pain to a genitofemoral nerve of a patient; and
an implantable electrical stimulation device that delivers electrical stimulation selected to alleviate pelvic pain to a genital nerve branch of at least one genitofemoral nerve of the patient.

29. The system of claim 28, wherein the drug is selected to alleviate pelvic pain including at least one of chronic testicular pain, post vasectomy pain, genitofemoral neuralgia, vulvodynia, and interstitial cystitis.
